(19) United States
(12) Patent Application Publication
(10) Pub. No.: US 2020/0148541 A1
(43) Pub. Date: May 14, 2020

(54) AN APPROACH TO A BOTTOM-UP SYNTHESIS OF NANOCARBONS

(71) Applicant: YISSUM RESEARCH DEVELOPMENT COMPANY OF THE HEBREW UNIVERSITY OF JERUSALEM LTD, Jerusalem (IL)

(72) Inventors: Ori GIDRON, Modiin (IL); Sunita PHATANGARE, Jerusalem (IL); Or DISHI, Mazkeret Batya (IL); Anjan BEDI, Jerusalem (IL)

(21) Appl. No.: 16/617,285

(22) PCT Filed: May 31, 2018
(86) PCT No.: PCT/IL2018/050593

§ 371 Date: Nov. 26, 2019

(51) Int. Cl. C01B 32/184; C01B 32/18; C07D 405/14

(52) U.S. Cl. CPC C01B 32/184; C07D 405/14; C01B 32/18

(57) ABSTRACT

Provided is a method for the synthesis of a π-conjugated system from oligofurans, under conditions involving cycloaddition.

### TECHNOLOGICAL FIELD

The invention generally concerns novel methodologies for the synthesis of nanocarbon structures.

### BACKGROUND

Nanocarbons, defined as nanometer-sized graphene allotropes, have dramatically changed the landscape of carbon-based materials in the past decades. The discoveries of fullerenes, carbon nanotubes, and the isolation of a single layer of graphene were major breakthroughs recognized by several Noble Prizes. These materials have opened doors to new technologies, with potential applications in many areas of materials science, including energy storage, catalysis, organic electronics, and spintronics.

Obtaining access to nanocarbons of defined size and shape allows chemists to tailor the electronic, magnetic, and optical properties of these materials. However, although many of these nanocarbon structures have been theoretically predicted, they remain yet to be synthesized.

Despite its many interesting electronic features, graphene itself lacks a measurable bandgap, which limits its application in organic electronics. In contrast, graphene nanoribbons (GNRs), which are segments of graphene with well-defined nanometric dimensions, display a controllable band gap, which makes them attractive as organic electronic components. Currently graphene-based materials are predominantly obtained using top-down methods (i.e., exfoliation from graphite), but these methods yield products lacking structural perfection.

Bottom up synthesis of nanocarbons remains a substantial challenge. In addition to their optoelectronic properties, several topologies of graphene nanoribbons are known to carry a spin current, and thus can be used as the active material in spintronic devices.

Carbon nanotubes (CNTs) can be considered as rolled-up structures of two-dimensional graphene nanoribbons. They are of great interest to materials chemistry, mostly owing to their outstanding physical and chemical properties and the wealth of potential applications in technology. Like the abovementioned GNRs, CNTs are not regarded as structurally pure molecules—which is one of the greatest obstacles currently limiting their application in molecular electronics.

The top-down synthesis of nanocarbons involves harsh reaction conditions. For example, CNTs are typically synthesized by arc discharge, laser ablation, or chemical vapor deposition methods. These methods produce CNTs with varying diameters and structures that cannot be completely isolated. GNRs, which can be produced by lithographic patterning of graphene or by the unzipping of CNTs, also typically exhibit a broad distribution of widths, lengths and edge structures.

The simplest component of carbon nanobelts (CNBs) is cycloparaphenylene (CPP), which consists exclusively of 1,4-connected phenylenes. This macrocycle represents the shortest possible cross-section of an armchair CNT. While CPP could be the ideal candidate for the bottom-up synthesis of CNTs, it is unfortunately unreactive towards Diels-Alder reaction, likely due to the high loss of aromatic stabilization energy that accompanies cycloaddition. For a specific dienophile, the tendency to undergo [4+2] cycloadditions in bay-regions increases from biphenyl to phenanthrene to perylene to bisanthene. This trend indicates that a nanobelts with perylene structural features (known as ‘Vogtle's belt’) should undergo a Diels-Alder cycloaddition.

In recent years, a new type of organic electronic materials, namely linear oligofurans (nF), was introduced, which display many advantages over the commonly explored oligothiophenes—greater solubility, rigidity/planarity, and strong fluorescence [1]. It was demonstrated that oligofurans have similar organic field-effect transistor (OFET) properties as benchmark oligothiophenes (nT), with the light emission that was often observed during device testing demonstrating their potential as organic light emitting transistors (OLETs) [2-3]. The Diels-Alder reactivity of oligofurans was also studied and demonstrated their highly regioselective and efficient reactions [4]. Following these results, it was clear that, unlike many π-conjugated systems (such as oligothiophenes and oligophenylenes) oligofurans are interesting not only for their electronic and optical properties, but also as substrates for the synthesis of novel π-conjugated systems, owing to their Diels-Alder reactivity.

### REFERENCE

[1] O. Gidron, Y. Diskin-Posner, M. Bendikov, J. Am. Chem. Soc. 2010, 132, 2148-2150.
[2] O. Gidron, A. Dadvand, Y. Sheynin, M. Bendikov, D. F. Perepichka, Chem. Commun. 2011, 47, 1976-1978.
[3] O. Gidron, A. Dadvand, E. W. H. Sun, I. Chung, L. J. W. Shimon, M. Bendikov, D. F. Perepichka, J. Mater. Chem. C 2013, 1, 4358-4367.
[4] O. Gidron, L. J. W. Shimon, G. Leitus, M. Bendikov, Org. Lett. 2012, 14, 502-505.
[5] J. Kromer, I. Rios-Carreras, G. Fuhrmann, C. Musch, M. Wunderlin, T. Debaerdemaeker, E. Mena-Osteritz, P. Bauerle, Angew. Chem. Int. Ed. 2000, 39, 3481-3486.

### SUMMARY OF THE INVENTION

Top-down synthetic methods for graphene nanoribbons (GNRs, FIG. 1, structure A) and carbon nanotubes (CNTs) yield products lacking structural perfection. While great advances in the field were achieved with the bottom-up synthesis of various GNRs, the synthesis of new nanocarbons remains a significant challenge. The synthesis of carbon nanobelts (CNBs, FIG. 1, structure B) is even more challenging: despite many attempts, no CNB has been reported to date. CNBs, which can be viewed as rolled-up structures of two-dimensional graphene nanoribbons, are considered as ‘seeds’ from which carbon nanotubes can be grown using the bottom-up approach—an important goal that is yet to be achieved. Currently most synthetic attempts apply Diels-Alder cycloaddition to the smallest constituent of these nanobelts, cycloparaphenylene (CPP, FIG. 1, structure C), which consists exclusively of 1,4-connected phenylenes. However, growing CPPs to nanobelts remains elusive, as CPPs are relatively inert towards Diels-Alder cycloaddition.

With the existing urgent need for novel synthetic methodologies that can produce new graphene-based materials essential, the inventors of the invention disclosed herein have developed a unique and efficient bottom-up approach for the production of such materials.

In recent years, the inventors had pioneered the synthesis of a new type of organic electronic material, namely, linear oligofurans (nF, FIG. 1, structure D), which display many advantages over the commonly explored oligothiophenes (nT, FIG. 1, structure E), such as greater solubility, rigidity/planarity, and strong fluorescence. Importantly, nF function as dienes, which can readily react in Diels-Alder cycloaddition, thus have great potential as reagents for other organic electronic materials.

As demonstrated herein, linear oligofurans can easily be converted to oligonaphthalenes (FIG. 2), and triphenylenes thereby demonstrating the conversion of one long conjugated backbone system to another, while maintaining conjugation. As oligoarenes can further be converted to graphene nanoribbons, these results highlight the use of oligofurans as ‘synthons’ for various nanocarbons.

The invention also constitutes an advance towards the introduction of macrocyclic oligofurans (nCF, FIG. 1, structure F). Such macrocycles can be considered infinite π-conjugated systems and therefore exhibit unique optical and electronic properties, which substantially differ from those of their linear analogues. While cyclic oligothiophenes (nCT, FIG. 1, structure G) are known, cyclic oligofurans are unknown.

The invention provides oligofurans as building blocks of a variety of novel and scientifically important materials. As used herein, the “oligofuran” is a linear chain of covalently associated furan rings or a cyclic macrocycle comprising covalently associated furan rings. The oligofuran, whether linear or cyclic substantially consists furan ring moieties. For the purposes herein, the oligofuran may comprise additional mid-chain or mid-cycle groups of ring systems that are not furan rings. However, unless specifically noted, the oligofuran consists furan ring moieties that may be ring-substituted, as recited herein. The oligofuran typically comprises two or more furan ring moieties. Thus, any use of an oligofuran or a structure comprising an oligofuran moiety may comprise two or more furan ring moieties. In some embodiments, the oligofuran comprises 2, 3, 4, 5, 6, 7, 8, 9, or 10 or more furan ring moieties.

In one aspect, the invention provides methods for converting an oligofuran to oligoarenes and a graphene nanoribbon. As shown in FIG. 3, the methodology provides the means to synthesize multi-substituted oligorylenes; synthesis of graphene segments; and synthesis of acene oligomers. The substituents shown in FIG. 3, are as disclosed herein, wherein each of R₁, R₂, R₃ and R₄ may independently be selected from hydrogen, C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl and C₆-C₁₀aryl. Variants R₅, R₆, R₇ and R₈ may each, independently, be similarly selected from hydrogen, C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl and C₆-C₁₀aryl. Integer n is between 2 and 100.

In another aspect, the invention provides a methodology for the synthesis of carbon nanobelts from macrocyclic oligofurans. The reaction of macrocyclic oligofurans with dienophiles such as benzyne precursors generates macrocyclic oligoarenes, which may be treated, e.g., by dehydrogenation, to yield the corresponding carbon nanobelts, as depicted for the purpose of exemplification in FIG. 4.

Thus, the invention generally provides a method for the synthesis of a π-conjugated system, the method comprising reacting an oligofuran with at least one dienophile (e.g., an aromatic dienophile which provides the ability to extend π-conjugation of the oligofuran), under the conditions described below, e.g., under conditions of Diels-Alder cycloaddition, as known in the art.

The invention further provides a method for the synthesis of a π-conjugated system, the method comprising reacting an oligofuran with at least one dienophile selected from an aromatic dienophile and a non-aromatic dienophile to convert each furan moiety in said oligofuran to a cycloadduct and reacting said cycloadduct to yield the π-conjugated system.

Where cycloaddition is used, it can be carried out under varying conditions, depending inter alia on the precursors used and the product to be achieved, by controlling cycloaddition conditions. Cycloaddition conditions can include varying temperatures, precursor ratios, solvents, pressures and reaction times. Typically, the cycloaddition may be carried out at a temperature above 0° C., or at a solvent boiling temperature, and under pressurized conditions to force interaction between the dienophile and the diene. In some cases, the reaction conditions may involve temperatures below room temperature (below 23-30° C.).

In some embodiments, the cycloaddition is carried out at a temperature between about 70° C. and 700° C., between 100° C. and 700° C., between 150° C. and 700° C., between 200° C. and 700° C., between 250° C. and 700° C., between 300° C. and 700° C., between 350° C. and 700° C., between 400° C. and 700° C., between 450° C. and 700° C., between 500° C. and 700° C., between 550° C. and 700° C., between 600° C. and 700° C., between 650° C. and 700° C., between 70° C. and 100° C., between 70° C. and 150° C., between 70° C. and 200° C., between 70° C. and 250° C., between 70° C. and 300° C., between 70° C. and 350° C., between 70° C. and 400° C., between 70° C. and 450° C., between 70° C. and 500° C., between 70° C. and 550° C., between 70° C. and 600° C., between 70° C. and 650° C., between 100° C. and 200° C., between 200° C. and 300° C., between 300° C. and 400° C., between 400° C. and 500° C., between 500° C. and 600° C. or between 600° C. and 700° C.

The cycloaddition may be carried out at ambient conditions (temperature and pressure) or may be carried out under conditions in which the temperature and/or the pressure are varied to achieve a maximum yield of cycloaddition. In some embodiments, the cycloaddition is carried out at atmospheric pressure. In some embodiments, the reaction is carried out at higher pressures, for example at between 2 and 500 bar, between 10 and 500 bar, between 50 and 500 bar, between 100 and 500 bar, between 150 and 500 bar, between 200 and 500 bar, between 250 and 500 bar, between 300 and 500 bar, between 350 and 500 bar, between 400 and 500 bar, between 450 and 500 bar, between 10 and 50 bar, between 10 and 100 bar, between 10 and 150 bar, between 10 and 200 bar, between 10 and 20 bar, between 20 and 30 bar, between 30 and 40 bar, between 40 and 50 bar, between 50 and 60 bar, between 60 and 70 bar, between 70 and 80 bar, between 90 and 100 bar, between 2 and 10 bar, between 2 and 20 bar, between 2 and 40 bar, between 2 and 50 bar or at 2, 3, 4, 5, 6, 7, 8, 9, or 10 bars.

The molar ratio between the oligofuran and the dienophile, e.g., aryne, may be stoichiometric or in excess. Depending on the length of the oligofuran, namely the number of furan ring moieties in the oligomer, the molar amount of the dienophile may be determined. For example, a molar ratio of 1 may designate a stoichiometric molar amount of dienophile, wherein the molar amount of dienophile is equal to the number of furan ring moieties in the oligofuran. As such, for example, an oligofuran having 6 furan rings would use 6 moles of the dienophile, constituting a molar ratio of 1. Thus, in some embodiments, the molar ratio may be equal to 1 or may be greater than 1, in which case the molar amount of dienophile is greater than the number of furan ring moieties in the oligofuran. In some embodiments, the dienophile is in an excess of 1.5, 2, 2.5, 3, 3.5, 4, 4.5, 5, 5.5, 6, 6.5, 7, 7.5, 8, 8.5, 9, 9.5, 10 . . . 100, 150 or a higher excess.

The cycloaddition can be carried out neat, in the absence of a liquid carrier, wherein the reaction mixture comprises the diene and dienophile, or may be carried out in a liquid medium, selected based, inter alai, on the boiling temperature of the liquid, its protonation ability, stability, reactability and other parameters known to a person versed in the art. The liquid medium may be made of a single liquid or a mixture of liquids and may be selected amongst aqueous liquids and non-aqueous liquids, e.g., or organic solvents. In some embodiment, the liquid is an aqueous medium, or a medium comprising any amount of water. In some embodiments, the liquid is an organic solvent selected amongst aromatic solvents, such as benzene, toluene, xylene, nitrobenzene, chlorobenzene, or from solvents such as dioxane, dimethylformamide (DMF), ethyl ether, hexane, cyclohexane, chloroform, dichloromethane, tetrahydrofuran (THF), ethyl acetate, acetone, acetonitrile and dimethyl sulfoxide (DMSO).

In some embodiments, the solvent is an alcohol such as methanol, ethanol, propanol, iso-propanol, butanol, pentanol, hexanol and others, used alone, in combination, or with water.

Depending on a combination of any of the cycloaddition parameters disclosed herein, and which are known in the art, the reaction times may vary between very short and several hours. In some cases, the cycloaddition may be completed after several minutes from the time the reaction begins, e.g., under the selected temperature and pressure, or may take as many as 48 hours to complete. While a maximum yield of the cycloaddition reaction is typically desired, in some cases, the maximum yield may not be reached. The progression of the reaction may be monitored, e.g., by TLC or chromatography, to determine the reacting time at which no more of the product is produced or when all reactants have been consumed.

In a typical cycloaddition reaction, a diene and a dienophile are reacted to yield an cycloadduct that, depending on the nature of both reactants, can subsequently undergo conversion to an aromatic system or to a conjugated system. The diene precursor or molecule is one having the structure —CRa═CRb—CRc═CRd-, wherein each Ra, Rb, Rc and Rd is a hydrogen or an atom or a group of atoms. The diene may be a cyclic diene such as furan or may be acyclic. The diene in the technology disclosed herein is the furan ring system making up the oligofuran precursor.

The dienophile is an alkene or an alkyne that is reactive toward a diene, e.g., furan, to provide a 4+2 cycloaddition product (Diels Alder Reaction). The dienophiles useful in the present methods include carbon-containing dienophiles such as alkenes, alkynes, nitriles, enol ethers, and enamines

In some embodiments, the dienophile is at least one aromatic ring system associated or having a reactive double or triple bond. In some embodiments, the aromatic ring system may or may not be substituted in order to introduce a variety of functional groups to the reaction adduct or the final product. In some embodiments, the dienophile is at least one aryne, e.g., benzyne (derived from benzene), phenanthryne (derived from phenanthrene), naphthalyne (derived from naphthalene) or a heteroaryne (derived from a heteroarene). In some embodiments, the aryne, e.g., benzyne, is derived from a phenyl ring, an anthracene ring system, a naphthalene ring system, phenanthrene, or any other arene or heteroarenes.

As may be understood form the disclosure provided herein, the diene system providing the π-conjugated system is oligofuran, which may be linear or cyclic in structure. Generally speaking, under the Diels-Alder cycloaddition conditions, and depending on the nature of the dienophile, a reaction product may be an exo- or an endo-adduct. The reaction may be carried out under specific conditions in order to control the nature of the product (or of the major product in cases both adducts are formed). Notwithstanding the end product, the conversion of the adduct to the aromatic conjugated system may be carried out by various deoxygenataion conditions as disclosed herein.

The methodology presented herein provides the ability to fine tune the structure or properties of highly complex as well as simply structured conjugated systems. Regioselective backbone transformation can be obtained by modifying the order of dienophile addition. The addition of one equivalent of maleimide to DM-3F (structure shown in FIG. 6) results in selective cycloaddition-aromatization to the terminal rings, and addition of two equivalents results in cycloaddition to the second terminal position, leaving the central furan ring to react with the stronger dienophile 1, eventually yielding oligoarene 7. The addition of one equivalent of maleimide to DM-3F, followed by two equivalents of 1 results in 8. The selectivity of this reaction was demonstrated by the subsequent addition of ethyl maleimide, methyl-maleimide and 1, yielding oligoarene 9, which consists of three different aryls, in a highly selective manner (FIG. 6). The simplicity of this approach contrasts sharply with the common method, involving transition metal catalized C—C coupling of pre-functionalized arenes.

Reacting long oligofurans with aryne precursors (FIG. 7) results in sequential Diels-Alder cycloadditions to the entire π-conjugated system. In this manner, oligofurans may be converted to contain, e.g., six units (6F) to the corresponding oligonaphthalenes (nNap), forming in this example up to 12 new C—C bonds in a single step. Unlike common arene-arene couplings, this method does not require prior functionalization or the use of transition metal catalysts.

The formation of a triphenylene dimer from 2F demonstrates the versatility of this transformation for other arenes. The X-ray structure of the product reveals that the transformation produced significant twisting of the triphenylene core. Tuning the reactivity and reaction sequence results in site-selective substituted aryl-formation.

The use of moderate dienophiles (even in large excess) may result in partial addition to the conjugated backbone. Thus, where relevant a strong dienophile was used to achieve full conversion of the π-conjugated backbone. Arynes were selected as dienophiles, with 2-(trimethylsilyl)phenyl trifluoromethanesulfonate (1) (FIG. 8A) as the benzyne precursor. The addition of 1 to 2F (nF, wherein n=2) with CsF at room temperature produced cycloadduct 2CA (nCA, wherein n=2) as the only product (FIG. 8A). The structure of 2CA was confirmed by X-ray crystallography, which revealed the syn conformation of the oxo-bridges. For the deoxygenation of oligo(epoxynaphthalene), the conditions used included the addition of trimethylsilyl chloride (TMSCl) and NaI in acetonitrile at 0° C. for 1.5 h, resulting in oligonaphthalenes (nNap) in moderate to high yields. Applying these conditions to 2CA produced 4,4′ -binaphthol as a major product, while the presence of methyl in the terminal position of DM-2F (DM-nF, wherein n=2) resulted in deoxygenation to yield bis-naphthalene DM-nNap. Series of α,α′-dimethyl substituted oligofurans DM-nF were prepared, where n=2−6 (FIGS. 8A and B). The synthesis of compound DM-6F-2C8 is depicted in FIG. 19.

Addition of 1 to DM-3F under the same conditions resulted in the formation of DM-3CA, which was subsequently deoxygenated to yield the ternaphthalene, DM-3Nap. Whereas DM-2Nap exists in two enantiomeric forms (M and P), the three connected naphthalene rings of DM-3Nap can exist in both the enantiopure M,M and P,P forms, as well as in its meso form (M, P, FIG. 1a). Indeed, EXSY-NMR (exchange spectroscopy) reveals two sets of interconverting peaks. Using VT-EXSY-NMR, the rotational barrier for DM-3Nap was extracted as ΔG^{‡}_{317K}=22.7 kcal mol⁻¹. This corresponds to values obtained for the racemization of 1,1′-binaphthyl (ΔG^{‡}_{317K}=24.4 kcal mol⁻¹). The M,M and P,P forms were separated from the meso (M,P) atropisomer using HPLC chromatography. The crystal structure, containing both M,M and P,P enantiomers of DM-3Nap, indicated that no reorganization takes place during the deoxygenation process and that the original connectivity is retained during backbone transformation. The packing is governed by weak C—H . . . π interactions (2.85 Å) between the π system of the terminal naphthalene unit and the C—H of the central naphthalene in the adjacent molecule.

To expand the scope of the transformation beyond naphthalene units, DM-2F was added to phenanthryne precursor 2 (10-trimethylsilylphenanthryl 9-trifluoromethanesulfonate), applying the same reaction conditions (conditions a and b are as indicated for FIG. 8A) for oligonaphthalenes (FIG. 9A). Consequent deoxygenation resulted in 4,4′-dimethyl-1,1′-bis(triphenylene) 4 with 64% yield. The compound was fully characterized by 2D NMR and its single crystal structure was also obtained, as structural data for triphenylene dimers appear to be unknown. The X-ray structure of 4 revealed that each naphthalene unit was twisted 24° out of planarity by steric repulsion from the adjacent triphenylene units.

As shown in FIG. 9B, an alternative method is provided for the dehydrogenative coupling to be achieved by the use of nickel-mediated aryl-aryl coupling reaction, the Yamamoto coupling. In general, by using larger aryne precursors, one can gain access to a large variety of GNRs by simply applying these three synthetic steps. The GNR may be endowed with functional groups to increase solubility and reduce aggregation such as alkyls, or with removable trialkylsilyls groups. The substituents provided in FIG. 9B are similar to those indicated herein, for relative positions.

Overall, the procedure of the invention is compatible with the synthesis of pre-connected polyaromatic systems. As the oxidative dehydrogenation of triphenylene units can result in the synthesis of polyaromatic hydrocarbons, the conversion of furans to oligo(triphenylenes) serves as a starting point for the synthesis of long graphene nanoribbons, as further detailed herein.

The full transformation of oligofurans to oligoarenes containing different aryl units in a regioselective manner is also provided herein. This was achieved by modifying the order of dienophile addition. As demonstrated (see for example FIGS. 5 and 6), the addition of maleimide to DM-3F results in selective cycloaddition-aromatization to the terminal rings and addition of 2 equivalents results in cycloaddition to the second terminal position, leaving the central furan ring to react with the stronger dienophile 1, eventually yielding oligoarene 7. The addition of 1 equivalent of maleimide to DM-3F, followed by 2 equivalents of 1 results in 8. The selectivity of this reaction was demonstrated by the subsequent addition of ethyl maleimide, methyl-maleimide and 1, yielding oligoarene 9, which consists of 3 different aryls, in a highly selective manner The simplicity of this approach contrasts sharply with the common method, involving transition metal catalysed C—C coupling of pre-functionalized arenes.

The oligofuran used in method of the invention may be selected amongst linear and cyclic covalently bonded furans having the structure:

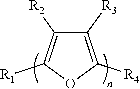

wherein

n is an integer defining the number of furan moieties that are bonded to each other (as shown, in some embodiments, each furan may be associated to at least one neighboring furan via the α-position);

each of R₁, R₂, R₃ and R₄ may independently be selected from hydrogen, C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; an alkyl stannyl; —CN; —CO₂H; —OH; —SH; —NR′R″R′″; —NO₂; (C₁-C₁₀alkyl)₃ Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counterion;

or

R₁ and R₂, or R₂ and R₃, or R₃ and R₄, together with the carbon atoms to which they are bonded form a 4-8-membered ring comprising between 0 and 3 double bonds, and/or between 0 and 3 heteroatoms selected from O, N, and S;

the 4-8 membered ring system being optionally substituted by at least one group selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; an alkyl stannyl; —CN; —CO₂H; —OH; —SH; —NR′R″R′″; —NO₂; (C₁-C₁₀alkyl)₃Si—; (C₁-C₁₀alkyl)₃Sn—;

trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion;

wherein at least one of R₁ and R₄ is a furan ring moiety of the oligofuran chain.

In some embodiments, where a 4-8 membered ring system may be formed, it is formed via groups R₂ and R₃, together with the carbon atoms to which they are bonded.

In some embodiments, where the oligofuran is linear, the number of furan rings may be between 3 and 500. Each furan ring is connected to another furan ring along the chain via a single covalent bond.

In some embodiments, the number of furan rings is at least 10, 20, 30, 40, 50, 60, 70, 80, 90, 100, 110, 120, 130, 140, 150, 160, 170, 180, 190 or at least 200. In some embodiments, the number of furan rings is at least 100, 200, 300, 400 or 500.

In some embodiments, the number of furan rings is between 3 and 50, between 3 and 45, between 3 and 40, between 3 and 35, between 3 and 30, between 3 and 25, between 3 and 20, between 3 and 15, between 3 and 10, between 3 and 9, between 3 and 8, between 3 and 7, between 3 and 6, between 3 and 5, between 5 and 45, between 5 and 40, between 5 and 35, between 5 and 30, between 5 and 25, between 5 and 20, between 5 and 15, between 5 and 10, between 10 and 45, between 10 and 40, between 10 and 35, between 10 and 30, between 10 and 25, or between 10 and 15.

In some embodiments, the number of furan rings is between 3 and 16, between 3 and 15, between 3 and 14, between 3 and 13, between 3 and 12, between 3 and 11, between 3 and 10, between 3 and 9, between 3 and 8, between 3 and 7 or between 3 and 6.

In such embodiments, where the oligofuran is linear, each of the end of chain furan may be substituted by one furan, constituting the extended part of the oligofuran, at one end, and by at least one group at the other end. The end groups may be same or different and may be selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion.

As used herein, the term “C₁-C₁₀alkyl” refers to a carbon group comprising between 1 and 10 carbon atoms, each carbon atom being associated with a neighboring carbon atom via a single covalent bond. The carbon atoms are typically substituted by hydrogen atoms or may be substituted. Exemplary alkyls include methyl, ethyl, propyl, isopropyl, isobutyl, n-butyl, sec-butyl, tert-butyl, iso-hexyl, heptyl octyl, nonyl, decyl and branched homologues thereof. In some embodiments, the C₁-C₁₀alkyl is an alkyl comprising between 1 and 10, 1 and 9, 1 and 8, 1 and 7, 1 and 6, 1 and 5, 1 and 4, 1 and 3, 2 and 10, 2 and 9, 2 and 8, 2 and 7, 2 and 6, 2 and 5, 2 and 4, 3 and 10, 3 and 9, 3 and 8, 3 and 7, 3 and 6, 3 and 5, 4 and 10, 4 and 9, 4 and 8, 4 and 7, 4 and 6, 5 and 10, 5 and 9, 5 and 8, 5 and 7, 6 and 10, 6 and 9, 6 and 8, 7 and 10, 7 and 9 or 8 and 10. In some embodiments, the number of carbon atoms is 1 or 2 or 3 or 4 or 5 or 6 or 7 or 8 or 9 or 10 carbon atoms.

The term “C₂-C₁₀alkenyl” refers to a carbon chain of from 2 to 10 carbon atoms and which contains 1 to 5 double bonds. The double bond may be along the alkenyl chain or may be a substituent that is attached to a main chain. In some embodiments, the C₂-C₁₀alkenyl is comprises between 2 and 10, 2 and 9, 2 and 8, 2 and 7, 2 and 6, 2 and 5, 2 and 4, 3 and 10, 3 and 9, 3 and 8, 3 and 7, 3 and 6, 3 and 5, 4 and 10, 4 and 9, 4 and 8, 4 and 7, 4 and 6, 5 and 10, 5 and 9, 5 and 8, 5 and 7, 6 and 10, 6 and 9, 6 and 8, 7 and 10, 7 and 9 or 8 and 10. In some embodiments, the number of carbon atoms is 2 or 3 or 4 or 5 or 6 or 7 or 8 or 9 or 10 carbon atoms.

The term “C₂-C₁₀alkynyl” refers to a carbon chain of from 2 to 10 carbon atoms and which contains 1 to 5 triple bonds. The triple bond may be along the alkynyl chain or may be a substituent that is attached to a main chain. In some embodiments, the C₂-C₁₀alkynyl is comprises between 2 and 10, 2 and 9, 2 and 8, 2 and 7, 2 and 6, 2 and 5, 2 and 4, 3 and 10, 3 and 9, 3 and 8, 3 and 7, 3 and 6, 3 and 5, 4 and 10, 4 and 9, 4 and 8, 4 and 7, 4 and 6, 5 and 10, 5 and 9, 5 and 8, 5 and 7, 6 and 10, 6 and 9, 6 and 8, 7 and 10, 7 and 9 or 8 and 10. In some embodiments, the number of carbon atoms is 2 or 3 or 4 or 5 or 6 or 7 or 8 or 9 or 10 carbon atoms.

The term “C₆-C₁₀aryl” refers to an aromatic monocyclic or multicyclic group containing from 6 to 10 carbon atoms. Aryl groups include unsubstituted or substituted fluorenyl, unsubstituted or substituted phenyl, and unsubstituted or substituted naphthyl.

The term “C₅-C₁₀ heteroaryl” refers to a monocyclic or multicyclic aromatic ring system of between 5 and 10 carbon atoms and one or more heteroatoms selected from nitrogen, oxygen and sulfur. The heteroaryl group may be optionally fused to a benzene ring. Heteroaryl groups include furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

An “alkyl stannyl” refers to an organic tin moiety having alkyl groups associated to a tin atom. Such moieties may be trialkyl stannyls, in which each of the alkyl groups may be the same or different. Each alkyl having between 1 and 10 carbon atoms, and selected as above (as defined for C₁-C₁₀alkyl). An example of an alkyl stannyl is the trialkyl (C₁-C₁₀alkyl)₃Sn—.

The group —NR′R″R′″ represents an amine group, or an ammonium group, wherein each of may be the same or different and may be selected as indicated herein. Where the group represents an amine, one of the substituting groups R′, R″ and R′″ is absent, e.g., in the form of —NR′R″. Where the group represents an ammonium group, the three substituting groups are present, the N atoms is charged and the group is associated with at least one counter-ion, which may be a negatively charged atom or a negatively charged group.

A halogen is an atom selected from I, Br, Cl and F. In some embodiments, the halide is Br.

In some embodiments, the oligofuran is α-oligofuran, namely each of the furan groups is bonded to another or to an end group via the carbon atom immediately associated with the furan oxygen atom.

In some embodiments, where the oligofuran is cyclic, the number of furan rings may be at least 4, 5, 6, 7, 8, 9, 10, 11, 12, 13, 14, 15, 16, 17, 18, 19 or 20. In some embodiments, the number of furan rings is 4, 5, 6, 7, 8, 9, 10, 11, 12, 13, 14, 15, 16, 17, 18, 19 or 20. In some embodiments, the number of furan rings is between 4 and 20, 4 and 19, 4 and 18, 4 and 17, 4 and 16, 4 and 15, 4 and 14, 4 and 13, 4 and 12, 4 and 11, 4 and 10, 4 and 9, 4 and 8, 4 and 7 or 4 and 6.

Thus, the oligo or polyfuran is of the general formula:

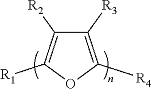

wherein one of R₁ and R₄ is a point of connectivity to another furan of the oligo or polyfuran and is therefore a furan ring, and the other of R₁ and R₄ is an end group selected as above. In some embodiments, the end group is H or an alkyl, as selected herein, for example being a C₁-C₁₀alkyl. The bond connecting each pair of furan rings is a single covalent bond.

The cyclicoligo or cyclicpolyfuran is of the general structure:

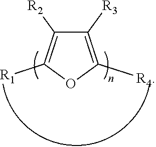

Where the cyclic system is formed through the alpha positions, each of R₁ and R₄ is a point of connectivity to another furan of the cyclicoligo or polyfuran ring system and is therefore a furan ring. The bond connecting each pair of furan rings is a single covalent bond.

The invention thus provides a cyclic furan oligomer or polymer comprising between 3 and 100 furan rings (n being between 3 and 100). In some embodiments, n is between 3 and 20.

The dienophile used in accordance with the invention may be selected from a variety of substituted or unsubstituted arynes and heteroarynes. In some embodiments, the aryne is at least one substituted or unsubstituted benzyne.

As used herein, the π-conjugated system being the product of the method, may be selected amongst oligoarenes, oligoacenes, graphene segments, carbon nanobelts and other π-conjugated system. In some embodiments, compounds produced according to methods of the invention are oligoarenes, oligoacenes, graphene segments, carbon nanobelts and specifically provided structures, as herein.

Thus, the invention further provides graphene segments of the structure:

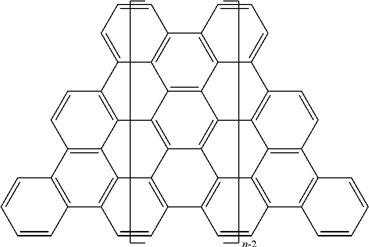

wherein

each peripheral carbon atom (where permitted) may be substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion; the substituting groups or atoms may or may not be the same; and

n is an integer between 2 and 100.

In some embodiments, where substitution is present, each substituting group may be different from the other. In some embodiments, all groups are the same.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

The invention further provides an oligoarylene of the structure:

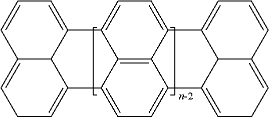

wherein

each peripheral carbon atom (where permitted) may be substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion; the substituting groups or atoms may or may not be the same; and

n is an integer between 2 and 100.

In some embodiments, each substituting group may be different from the other.

In some embodiments, all groups are the same.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

In some embodiments, n is different from 2.

The invention also provides an oligoacene of the structure:

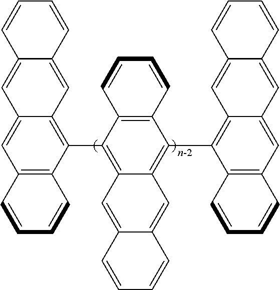

wherein

each peripheral carbon atom (where permitted) may be substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl, C₆-C₁₀aryl; wherein in case of charged amine groups, the group is associated with at least one counter-ion; the substituting groups or atoms may or may not be the same; and

n is an integer between 2 and 100.

In some embodiments, each substituting group may be different from the other. In some embodiments, all groups are the same.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

In some embodiments, n is different from 2.

In some embodiments, the oligoacenes are selected from substituted tetracenes, pentacene and hexacenes.

Cyclic oligofurans may be synthesized using the method introduced by Bauerle and shown in FIG. 10, Route A [5], by forming alkyne-Pt macrocycles followed by reductive elimination to obtain diacetylene macrocycles, which are consequently oxidized to yield macrocyclic oligofurans, or by an alternative pathway which involves the Friedel-Crafts acylation of furans with acetyl chloride followed by the Paal-Knorr reaction of the diketone linker (FIG. 10, Route B).

The cyclic oligofurans synthesized may be subjected to various aryne precursors, eventually resulting in n-cycloarenes by applying the previously-described cycloaddition-deoxygenation procedure (FIG. 11). Importantly, this synthetic strategy provides easy access to a wide variety of n-cycloarenes by simply introducing the macrocyclic oligofurans to different arene precursors. In this manner, the very limited family of n-cycloarenes may be expended.

The conversion of long oligofurans to multi-substituted GNRs, highly substituted oligorylenes and higher graphene segments have been prepared following the route presented in FIGS. 12A and B. In the first step, a library of oligofurans and benzyne precursors are synthesized, aiming for graphene nanoribbons with specific features such as donor-acceptors, specific shapes or bulky group to avoid aggregation. FIG. 12B, Route A, describes the synthesis of polysubstituted graphene nanoribbon using the cycloaddition-deoxygenation in the first step, followed by dehydrogenative coupling to yield oligorylenes. As in some instances the coupling can prove to be particularly challenging, often prone to reorganization, alternative pathways, including oxidative and reductive dehydrogenative couplings have been considered. An alternative pathway (Route B) was proposed, where the key step is a thermodynamic ring closing metathesis (RCM).

Thus, the invention provides a method for the synthesis of a π-conjugated system, the method comprising reacting an oligofuran with at least one dienophile selected from an aromatic dienophile and a non-aromatic dienophile to convert each furan moiety in said oligofuran to a cycloadduct and reacting said cycloadduct to yield the π-conjugated system.

In some embodiments, the cycloadduct is an extended cycloadduct prepared by reacting the cycloadduct with at least one diene, prior to conversion to the π-conjugated system.

In some embodiments, the at least one diene is a substituted or unsubstituted furan compound.

In some embodiments, the furan compound is a phenyl substituted furan.

In some embodiments, the furan compound is diphenyl-isobenzofuran.

In some embodiments, the cycloadduct is an oxo- or syn-cycloadduct, such that each cycloadduct is in the form of an oxo-cyclocadduct or a syn-cycloadduct.

In some embodiments, the cycloadduct is converted to the π-conjugated system by at least one of deoxygenation, hydrogentation, and oxidative hydrogentaion.

In some embodiments, the at least one dienophile is a mixture of two or more different dienophiles used as a mixture of dienophiles or reacted one after the other.

In some embodiments, the at least one dienophile is prepared in situ.

In some embodiments, the at least one dienophile is selected from substituted or unsubstituted arynes and heteroarynes.

In some embodiments, the aryne or heteroaryne is a multicyclic-aryne or multicyclic-heteroaryne.

In some embodiments, the aryne is selected from benzyne, phenanthryne and naphtharyne, each being optionally substituted.

In some embodiments, the aryne is at least one substituted or unsubstituted benzyne or a substituted or unsubstituted phenanthryne.

In some embodiments, the reaction between the oligofuran and the at least one dienophile is carried under Diels-Alder cycloaddition conditions.

In some embodiments, the π-conjugated system is selected amongst oligoarenes, oligoacenes, graphene segments and carbon nanobelts.

In some embodiments, the oligofuran is selected amongst linear oligofurans and cyclic oligofurans.

In some embodiments, the oligofuran is of the structure:

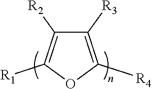

wherein

n is an integer defining the number of furan moieties that are bonded to each other;

each of R₁, R₂, R₃ and R₄ is independently selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen,

wherein each of R′, R″ and R′″ is the same or different and is selected from hydrogen; C₁-C₁₀alkyl ; C₂-C₁₀alkenyl; C₂-C₁₀alkynyl; and C₆-C₁₀aryl;

or

R₁ and R₂, or R₂ and R₃, or R₃ and R₄, together with the carbon atoms to which they are bonded form a 4-8-membered ring comprising between 0 and 3 double bonds, and/or between 0 and 3 heteroatoms selected from O, N, and S;

the 4-8 membered ring system being optionally substituted by at least one group selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; and C₆-C₁₀aryl;

wherein at least one of R₁, R₂, R₃ and R₄ is a furan ring moiety of the oligofuran chain.

In some embodiments, at least one of R₁ and R₄ is a furan ring moiety.

In some embodiments, each of R₁ and R₄ is a furan ring moiety and n is between 1 and 200.

In some embodiments, one of R₁ and R₄ is a furan ring moiety and the other of R₁ and R₄ is selected from hydrogen; C₁-C₁₀alkyl; —CN; —CO₂H; (C₁-C₁₀alkyl)₃Sn—; and a halogen.

In some embodiments, one of R₁ and R₄ is a furan ring moiety and the other of R₁ and R₄ is selected from hydrogen; an alkyl stannyl and a halogen.

In some embodiments, the 4-8 membered ring system is formed via groups R₂ and R₃, together with the carbon atoms to which they are bonded.

In some embodiments, the oligofuran is linear, comprising between 3 and 500 furan ring moieties, each connected to another via an α-carbon.

In some embodiments, the number of furan rings is at least 10, 20, 30, 40, 50, 60, 70, 80, 90, 100, 110, 120, 130, 140, 150, 160, 170, 180, 190 or at least 200.

In some embodiments, the number of furan rings is between 3 and 50, between 3 and 45, between 3 and 40, between 3 and 35, between 3 and 30, between 3 and 25, between 3 and 20, between 3 and 15, between 3 and 10, between 3 and 9, between 3 and 8, between 3 and 7, between 3 and 6, between 3 and 5, between 5 and 45, between 5 and 40, between 5 and 35, between 5 and 30, between 5 and 25, between 5 and 20, between 5 and 15, between 5 and 10, between 10 and 45, between 10 and 40, between 10 and 35, between 10 and 30, between 10 and 25, or between 10 and 15.

In some embodiments, the number of furan rings is between 3 and 16, between 3 and 15, between 3 and 14, between 3 and 13, between 3 and 12, between 3 and 11, between 3 and 10, between 3 and 9, between 3 and 8, between 3 and 7 or between 3 and 6.

In some embodiments, the oligofuran is linear, each end-of- the chain furan having an a -carbon substituted by a group selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; and C₆-C₁₀aryl.

In some embodiments, the oligofuran is α-oligofuran.

In some embodiments, the oligofuran is a cyclic oligofuran, comprising at least 4, 5, 6, 7, 8, 9, 10, 11, 12, 13, 14, 15, 16, 17, 18, 19 or 20 furan ring moieties.

In some embodiments, the number of furan rings is 4, 5, 6, 7, 8, 9, 10, 11, 12, 13, 14, 15, 16, 17, 18, 19 or 20.

In some embodiments, the number of furan rings is between 4 and 20, 4 and 19, 4 and 18, 4 and 17, 4 and 16, 4 and 15, 4 and 14, 4 and 13, 4 and 12, 4 and 11, 4 and 10, 4 and 9, 4 and 8, 4 and 7 or 4 and 6.

In some embodiments, the cyclic oligofuran is of the structure:

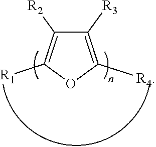

wherein each of n, R₁, R₂, R₃ and R₄ are as defined herein.

In some embodiments, the π-conjugated system is of the structure:

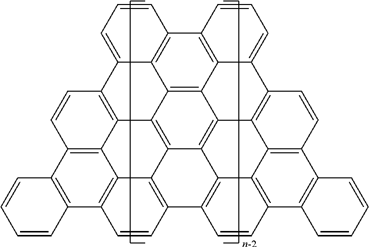

wherein

each peripheral carbon atom is substituted with a group or an atom selected from hydrogen; C₁-C₁₀alkyl ; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and n is an integer between 2 and 100.

In some embodiments, each substituting group is same or different from the other.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

In some embodiments, the π-conjugated system is of the structure:

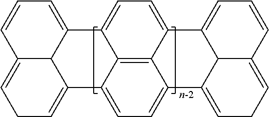

wherein

each peripheral carbon atom is substituted with a group or an atom selected from hydrogen; C₁-C₁₀alkyl ; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and

n is an integer between 2 and 100.

In some embodiments, each substituting group is the same of different from the other.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

In some embodiments, the π-conjugated system is of the structure:

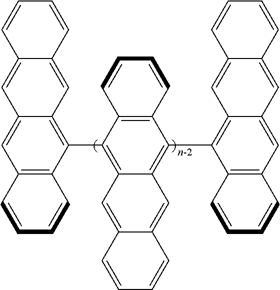

wherein

each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and

n is an integer between 2 and 100.

In some embodiments, each substituting group is the same or different from the other.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

In some embodiments, n is different from 2.

In some embodiments, the conjugated system is selected from substituted tetracenes, pentacene and hexacenes.

The invention further provides a compound having the structure:

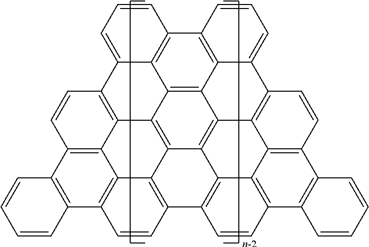

wherein

each peripheral carbon atom is substituted with a group or an atom selected from hydrogen; C₁-C₁₀alkyl ; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and

n is an integer between 2 and 100.

In some embodiments, each substituting group is same or different from the other.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

The invention further provides a compound of the structure:

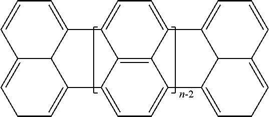

wherein

each peripheral carbon atom is substituted with a group or an atom selected from hydrogen; C₁-C₁₀alkyl ; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and

n is an integer between 2 and 100.

In some embodiments, each substituting group is the same of different from the other.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 2 and 16.

The invention also provides a compound of the structure:

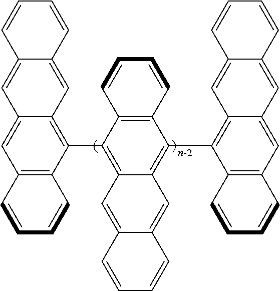

wherein

each peripheral carbon atom is substituted with a group or an atom selected from hydrogen; C₁-C₁₀alkyl ; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and

n is an integer between 3 and 100.

In some embodiments, each substituting group is the same or different from the other.

In some embodiments, n is between 2 and 95, 2 and 90, 2 and 85, 2 and 80, 2 and 75, 2 and 70, 2 and 65, 2 and 60, 2 and 55, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5 or 2 and 4.

In some embodiments, n is between 3 and 16.

In some embodiments, the compound is selected from substituted tetracenes, pentacene and hexacenes,

In some embodiments, the oligofuran is prepared by reacting a halo-substituted furan or a halo-substituted oligofuran with an alkyl stannyl substituted furan or oligofuran under conditions affording said oligofuran.

In some embodiments, the conditions involve use of a catalyst, optionally a Pd catalyst.

The invention further provides a method for the synthesis of a cyclic oligofuran, the method comprising:
reacting an alkyne-substituted oligofuran in the presence of a metal complex to afford an alkyne-metal macrocycle,
treating said macrocycle to obtain a di-acetylene macrocycle and
oxidizing said di-acetylene macrocycle to afford the cyclic oligofuran.

In some embodiments, the cyclic oligofuran comprises between 3 and 8 furan ring moieties.

In some embodiments, the end-of-chain furan ring moieties are substituted by an acetylene group.

In some embodiments, the metal complex is a Pt complex.

In some embodiments, the alkyne metal macrocycle is an alkyne-Pt macrocycle.

In some embodiments, the macrocycle is reductively eliminated to yield the di-acetylene macrocycle.

The invention further provides a method for the synthesis of a cyclic oligofuran, the method comprising:
reacting furan with succinyl chloride to afford a linear furan dimer, wherein each furan is separated by a succinyl moiety,
optionally repeating the reaction to afford higher homologs of the furan dimer,
reacting the furan dimer or higher homolog thereof with succinyl chloride under conditions permitting cyclization into a cyclic macrocycle, and
transforming each succinyl moiety of the cyclic macrocycle to a furan, thereby providing the cyclic oligofuran.

The invention further provides a method for the synthesis of a cyclic oligofuran, the method comprising:
reacting a linear oligofuran with succinyl chloride to afford a linear oligofuran dimer, wherein each oligofuran is separated by a succinyl moiety,
optionally repeating the reaction to afford higher homologs of the dimer,
reacting the dimer or higher homolog thereof with succinyl chloride under conditions permitting cyclization into a cyclic macrocycle, and
transforming each succinyl moiety of the cyclic macrocycle to a furan, thereby providing the cyclic oligofuran.

In some embodiments, the linear oligofuran comprises between 3 and 8 furan ring moieties.

In some embodiments, the linear oligofuran dimer comprises two oligofuran moieties separated by a succinyl moiety, each of the two oligofuran moieties, independently, comprises between 3 and 8 furan ring moieties.

In some embodiments, the method further comprising reacting the dimer with succinyl chloride and optionally in the presence of the linear oligofuran to obtain a linear oligofuran trimer or tetramer or a higher homolog.

In some embodiments, the method further comprising reacting the dimer, trimer, tetramer or higher homolog with a succinyl chloride under conditions permitting transformation of the linear oligofuran dimer, trimer, tetramer or higher homolog into a cyclic macrocycle.

In some embodiments, the conditions permitting transformation include addition of the succinyl chloride under dilution conditions.

In some embodiments, the succinyl moieties are transformed into furan moieties in the presence of acid.

Also provided is a cyclic oligofuran comprising between 4 and 20 furan ring moieties, each furan ring moiety being covalently associated to another via the furan α-carbon atoms.

In some embodiments, the oligofuran having the structure

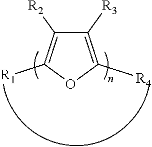

wherein

each of R₁ and R₄ is a point of connectivity to another furan of the cyclic oligofuran ring system,

R₂ and R₃ is selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl and C₆-C₁₀aryl; and

wherein n is an integer between 4 and 20.

In some embodiments, the compound comprises 6 furan ring moieties, wherein each of the furan ring moieties is optionally substituted.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-G provide structures of compounds A-G discussed in the present application.

FIG. 2 is a schematic representation of certain embodiments of the methodology of the invention.

FIG. 3 depicts conversion of oligofurans to oligoacenes and GNRs, wherein the various R groups may be the same or different or are selected as disclosed herein.

FIG. 4 provides an exemplary pathway for the production of cycloarenes and carbon nanobelts according to the invention.

FIG. 5 provides a schematic representation of substituted arene synthesis from oligofurans, using different types of dienophiles. The letters m and s in parentheses stand for moderate and strong dienophile strength, respectively.

FIG. 6 depicts synthesis of substituted arenes from oligofuran DM-3F and dienophiles: 2-(trimethylsilyl)phenyl trifluoromethanesulfonate (1) as the benzyne precursor; 5 and 6 are N-methylmaleimide and N-ethylmaleimide, respectively.

FIG. 7 demonstrates regioselective transformation of long oligofurans to oligoarenes as described herein.

FIGS. 8A-B are synthetic procedures for the transformation of oligofurans to oligonaphthalenes. For both Figures, reagents and conditions: (a) 1, acetonitrile, CsF, RT; (b) Trimethylsilylchloride, NaI, acetonitrile, 0° C.→RT. DM=dimethyl. For DM-3F n=2, 3, 4, 6. For nF: n=2, 6.

FIGS. 9A-B present exemplary synthesis of bis(triphenylene) from DM-2F (FIG. 9A). Reagents and conditions: (a) CsF, acetonitrile, RT; (b) Trimethylsilylchloride, NaI, acetonitrile, 0° C.→RT. FIG. 9B demonstrates conversion of nF to graphene nanoribbos. Reagents and conditions: (a) i. CsF. ii. NaI, SiMe₃Cl. (b) Dehydrogenative coupling (K⁰; FeCl₃; AlCl₃). (c) [Ni(cod)₂], 1,5-cyclooctadiene, 2,2′-bipyridyl. cod=1,5-cyclooctadiene. Groups X are equivalent to variants R as used herein, and integers n are as defined herein.

FIG. 10 provides exemplary routes for the synthesis of cyclic oligofurans.

FIG. 11 provides reaction of oligofurans, similarly applicable to linear and cyclic oligofurans, with benzyne, demonstrating their conversion to oligonaphthyls via cycloadduct 1. Reagents and conditions: (a) 2-(trimethylsilyl)phenyl trifluoromethanesulfonate, CsF, acetonitrile. (b) NaI, SiMe₃Cl. R=n-hexyl.

FIGS. 12A-B provide synthetic pathways to oligoacenes and graphene nanoribbons fromoligofurans. FIG. 12A provides general synthetic pathways and FIG. 12B provides synthetic pathway to oligoacenes and graphene nanoribbons from nF. Variants R₁, R₂, R₃ and R₄ are selected as recited herein regarding the equivalent variants in oligofurans of the invention. Reagents and conditions: (a) i. CsF. ii. NaI, SiMe₃Cl. (b) Dehydrogenative coupling (K⁰ or FeCl₃ or AlCl₃). (c) i. DMAD. ii. MeONa, MeOH. iii. Cu, heat (decarboxylation). (d) Grubbs' second-generation catalyst, PhMe, reflux. DMAD=dimethyl acetylenedicarboxylate.

FIG. 13 demonstrates conversion of a terafuran 4F to graphene nanoribbon 4. Reagents and conditions: (a) i. CsF. ii. NaI, SiMe₃Cl. (b) AlCl₃, H⁺. Various substituting groups are as recited herein with respect to the oligofuran.

FIG. 14 demonstrates synthesis of oligotetracene 8 by the reaction of cycloadduct 6 with isobenzofuran 5. Reagents and conditions: (a) Acetonitrile, heat. (b) p-toluenesulfonic acid, toluene.

FIG. 15 provides synthetic pathways for 8CF; reagents and conditions: (a) Pt(dppp)₂Cl₂, CuI (10% mol), NEt₃; (b) I₂ (2 equiv.), THF, 60° C. (c) CuI, 1,10-phenanathroline, KOH, H₂O, DMSO. (d) AlCl₃, ClC(O)CH₂CH₂C(O)Cl. (e) AlCl₃, ClC(O)CH₂CH₂C(O)Cl, high dilution. (f) HCl, heat. dppp=1,3-Bis(diphenylphosphino)propane, THF=tetrahydrofuran, DMSO=dimethylsulfoxide, NEt₃=trimethylamine The various substituting groups are as recited herein with reference to the oligofuran.

FIG. 16 demonstrates conversion of cyclic oligofuran 6CF to 6-cyclonapthalene 16, and to a soluble Vogtle's belt 17. Reagents and conditions: (a) i. CsF. ii. NaI, SiMe₃Cl. (b) AlCl₃, H⁺. The various substituting groups are as recited herein with reference to oligofuran.

FIG. 17 demonstrates conversion of macrocyclic furans to carbon nanobelts. Reagents and conditions: (a) i. CsF. ii. NaI, SiMe₃Cl. (b) AlCl₃, H⁺.

FIG. 18 is a schematic representation of growth of 20 to long CNTs by the cycloaddtion of 15 with 6CF.

FIG. 19 provides synthesis of DM-6F-2C8 (utilized as depicted in FIG. 8B).

FIGS. 20A-D provide calculated (FIG. 20A) HOMO-LUMO gaps and (FIG. 20B) inter-ring C—C bond lengths for nCF and nCT. FIG. 20C provides calculated structures of 6CF and 6CT. FIG. 20D provides the value for the exo angles of furan (125°) is closer to that of a hexagon and octagon, compared with that of thiophene (150°). All calculations were performed at the (DFT/B3LYP/6-311G(d)) level of theory.

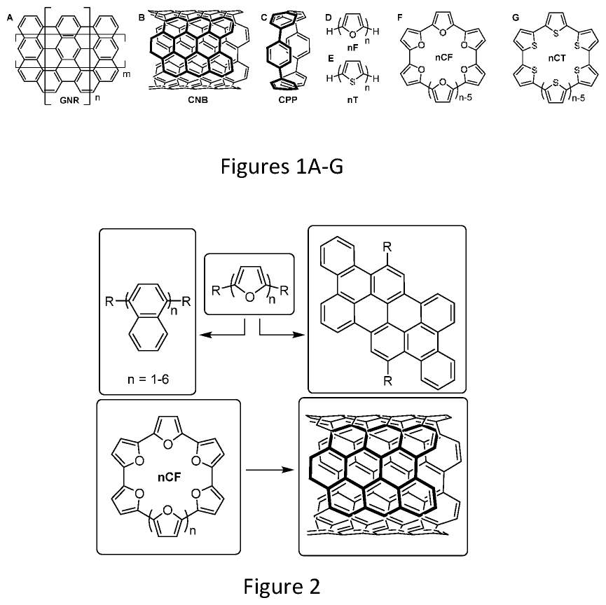

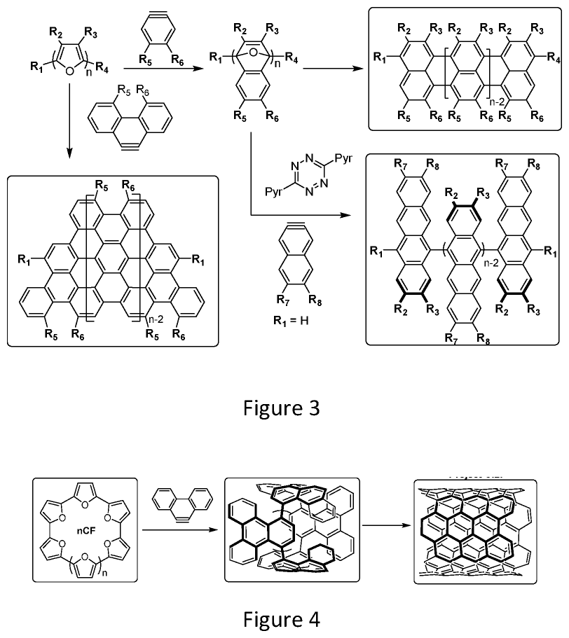

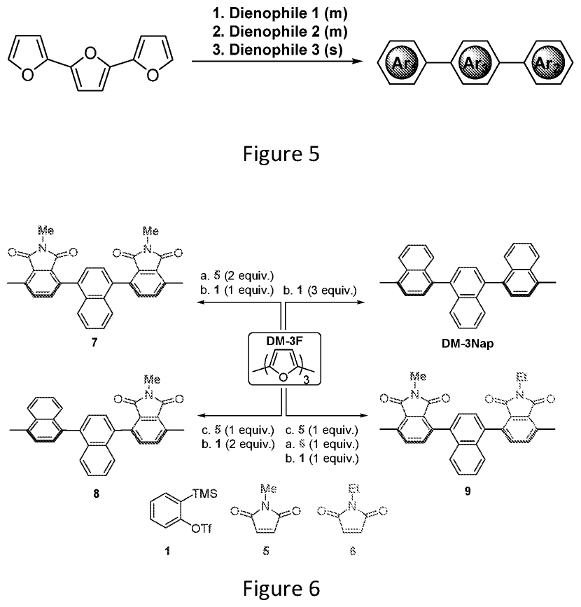

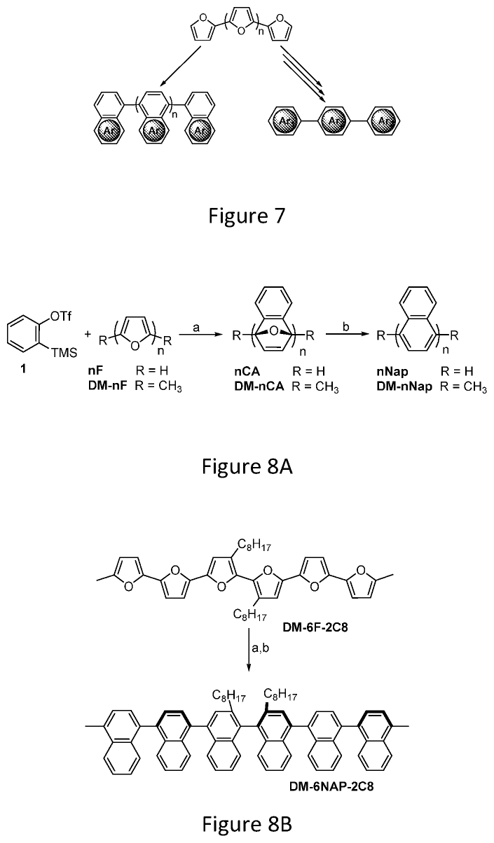

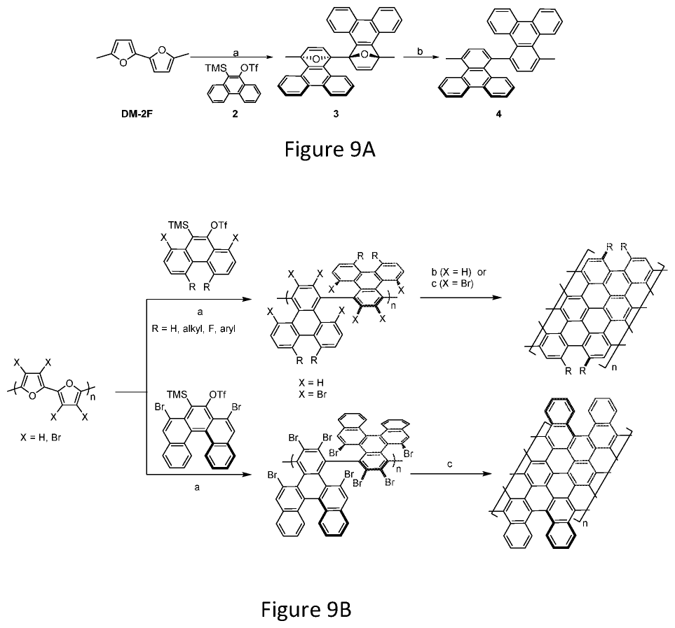

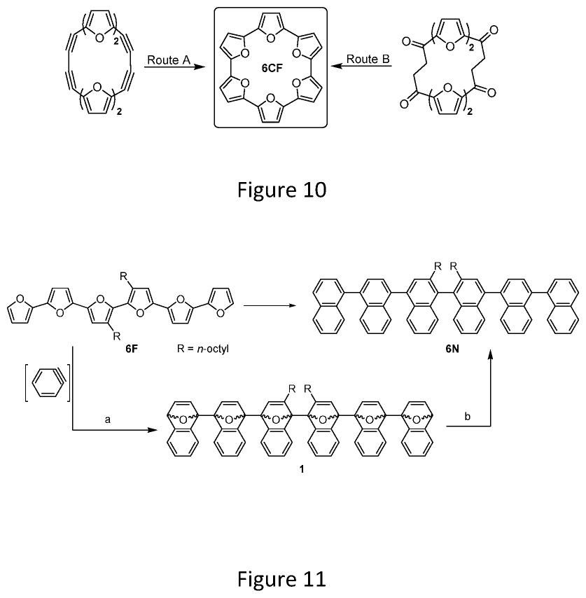

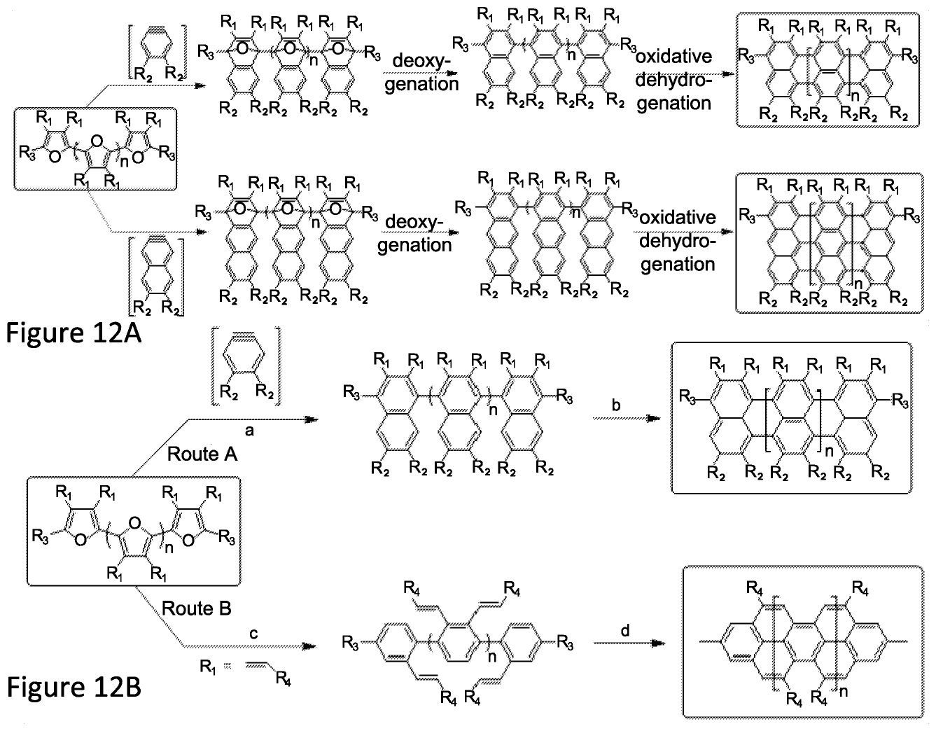

### DETAILED DESCRIPTION OF EMBODIMENTS

In a study leading to the technology disclosed herein, linear oligofurans were reacted with a benzyne precursor, resulting in direct conversion to the cycloadduct 1. Consequent deoxygenation of the cycloadduct resulted in clean conversion to oligonaphthalene 6N. This one-pot reaction demonstrated the direct conversion of one long conjugated system to another. As oligoarenes can undergo dehydrogenation to yield graphene nanoribbons, these results highlight the potential contribution of oligofurans to the field of nanocarbons.

Combining the conversion of linear furans to graphene-based materials with the introduction of macrocyclic furans yields a new bottom-up synthetic approach in which macrocyclic oligofurans are reacted with arynes to produce cycloarenes, eventually leading to the synthesis of the first carbon nanobelts and carbon nanotubes.

The disadvantage of the current synthetic approach to CPPs, which employs reductive aromatization of a cyclohexadiene moiety, is its limited versatility. For example, in order to obtain different substituted cycloarenes, one must initiate the synthesis with a different arene macrocycles each time. In this respect, the introduction of a divergent approach using a single intermediate from which various cycloarenes can be easily obtained is highly desirable.

The method of the invention possesses two great advantages over the commonly applied pathway for obtaining CPPs. Whereas different-sized macrocycles are required to synthesize different cycloarenes over multiple steps, a single cyclic furan can serve as a starting point for various cycloarenes in a single step (cycloaddition and deoxygenation), in this way enabling the rapid development of different cycloarenes that can be converted to a variety of carbon nanobelts.

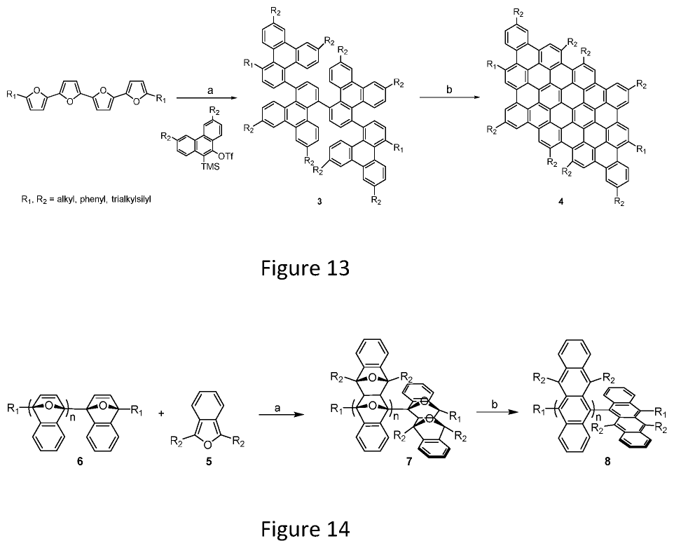

The synthesis of very large graphene nanoribbons commonly involves a significant number of steps, and is often limited by the insolubility of the reaction intermediates. The use of different aryne precursors allows the introduction of different oligoarenes with various sizes, eventually leading to GNRs having specific and controllable sizes and shapes. An example for such a procedure is demonstrated by the cycloaddition reaction between 4F and a phenanthryne precursor to afford oligoaryl 3, whose oxidative dehydrogenation will afford GNR 4 (FIG. 13). In general, by using larger aryne precursors, one can gain access to a large variety of GNRs by simply applying these three synthetic steps. Importantly, the GNR may be endowed with functional groups to increase solubility and reduce aggregation such as alkyls, or removable trialkylsilyls groups.

Introducing oligomers of substituted tetracenes and pentacenes can make a significant contribution. The cycloadduct itself can react as a dienophile with various dienes, which enables a lateral expansion of the conjugated system (via an extended cycloadduct). A diene that can very plausibly undergo this cycloaddition is diphenyl-isobenzofuran 5 (FIG. 14). Indeed, the reaction of such a diene with a monomeric analogue of 6 is known to result in the synthesis of various tetracenes. Thus, cycloaddition of 6 with 5 esults in ditetracene 8. A two-cycloaddition/deoxegenation process yields “rubrene-like” oligomers (FIG. 14).

In many cases, the physical properties of linear oligomers are influenced by undesired chain-end effects. In this respect, corresponding fully π-conjugated macrocycles represent model systems that combine the ‘infinite’ defect-free π-conjugated chain of an idealized polymer with the advantage of a structurally well-defined oligomer, while excluding perturbing end-effects. This renders them interesting candidates for various future applications in organic and molecular electronics and for the study of host—guest interactions, aggregation, and self-assembly on surfaces.

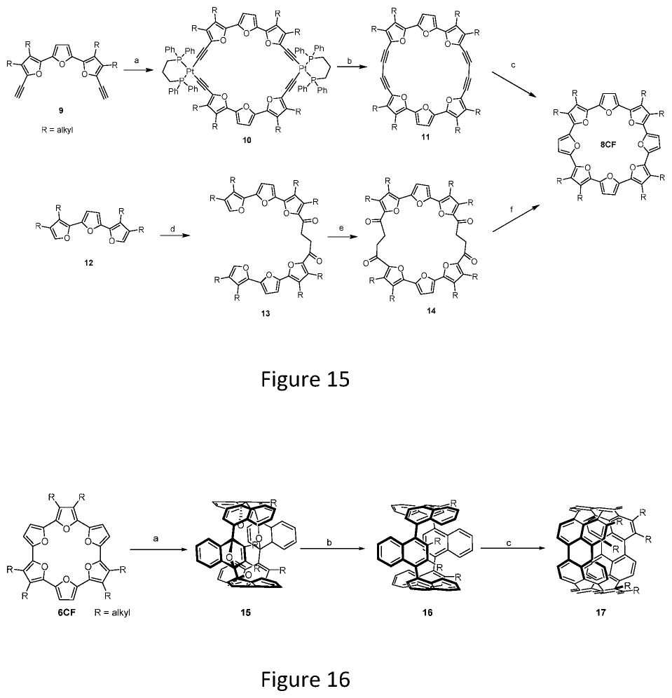

In order to synthesize macrocyclic oligofurans, several synthetic pathways are explored, beginning with the two shown in FIG. 15 for the synthesis of 8CF. The first pathway, which was introduced by Bauerle involves the formation of alkyne-Pt macrocycles followed by reductive elimination to obtain diacetylene macrocycles. The diacetylene moiety is consequently oxidized to yield macrocyclic oligofuran 8CF. An alternative pathway involves the Friedel-Crafts acylation of terfuran 12 with acetyl chloride followed by an additional acylation at high dilution to yield macrocycle 14. A Paal-Knorr reaction will transform the diketone moieties to furans, eventually resulting in 8CF.

The macrocyclic oligofurans synthesized is reacted with various aryne precursors to form macrocyclic cycloadducts. The simplest example involves cycloaddition with benzyne and results in cycloadduct 15 (FIG. 16), which can be deoxygenated to form n-cyclonaphthalene 16, in a manner similar to that successfully applied to liner oligofurans in the previously-described cycloaddition-deoxegenation procedure. The R groups on 6CF are as selected herein, and may particularly be selected amongst alkyls, for increased solubility of the resulting CNB 17.

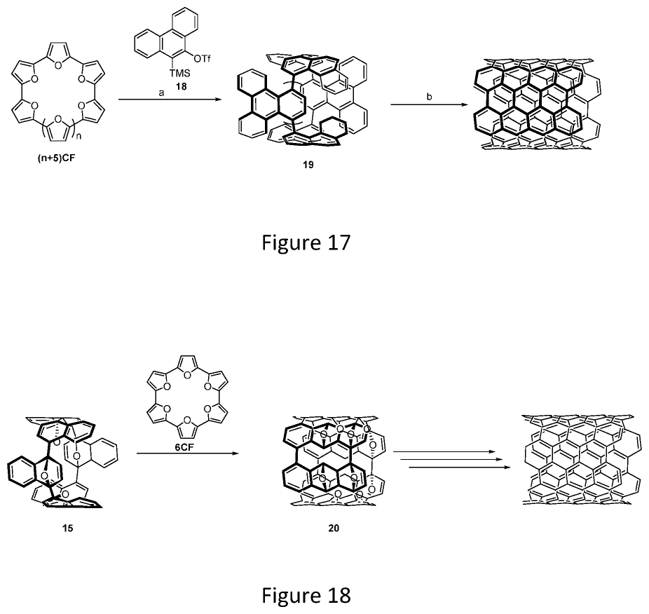

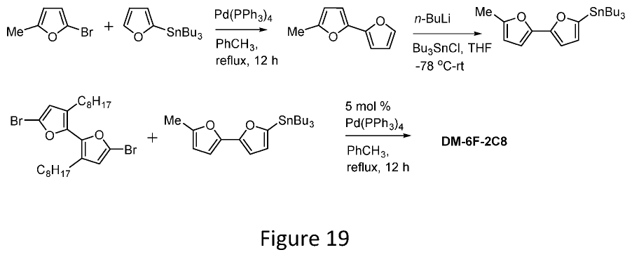

Importantly, this synthetic strategy provides easy access to a wide variety of cycloarenes, by simply introducing the macrocyclic oligofurans to different aryl precursors. While macrocyclic arene 19 obtained from the addition of phenanthryne precursor 18 was expected to result in the anti-conformation, the oxidative dehydrogenation in this case was expected to be favorable because of the vicinity of the phenyl rings, as depicted in FIG. 17.

In a similar manner as previously observed for monomeric furans, cycloadduct 15 exhibited affinity towards dienes, thus resulting in further expansion of the carbon nanobelts. The reactivity of 15 was studied through its reaction with various dienes. Cycloaddition results in compound 20, which can again react with an additional segment of 6CF, resulting in a tandem Diels-Alder cycloaddition and eventually growing long CNTs in a bottom-up approach (FIG. 18). In order to increase the reactivity of 6CF, electron-donating groups such as methoxy may be added to the β position, which are expected to significantly increase its reactivity towards dienophiles.

The resulting CNBs may be endowed with soluble groups, resulting from either the macrocycle or from the benzyne precursors. Alkyl groups should increase the solubility of the CNBs.

### Experimental Section

¹H and ¹³C NMR spectra were recorded in solution on a Briicker-AVIII 400 MHz and 500 MHz spectrometer using tetramethylsilane (TMS) as the external standard. Chemical shifts are expressed in δ units. High resolution mass spectra were measured on a HR Q-TOF LCMS and Waters Micromass GCT Premier Mass Spectrometer using ESI and field desorption (FD) ionization. The spectra were recorded using chloroform-d, 1,1,2,2-tetrachloroethane-d₂, DMSO-d₆ as solvents. Flash chromatography (FC) was performed using CombiFlash SiO₂ columns. N-methyl maleimide, N-ethyl maleimide and 2-methylfuran were purchased from Sigma-Aldrich. 2-(Trimethylsilyl)phenyl trifluoromethanesulfonate and 10-trimethylsilylphenanthryl 9-trifluoromethanesulfonate were purchased from Alfa Aesar and abcr respectively. Compounds DM-3F, 2,8-dibromobifuran and 3,3′-octyl-2,2′-bifuran were synthesized according to literature procedure.

### Synthetic Procedure

5,5′-dimethyl-2,2′-bifuran (DM-2F)

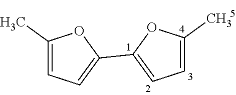

A three neck RB was charged with 2-methylfuran (5 g, 60.90 mmol) and THF (90 mL) (freshly distilled). After it was cooled to −78° C., a solution of n-BuLi (38.0 mL, 1.6 M, 60.90 mmol) was added dropwise. After addition, the mixture was warmed slowly to room temperature and stirred for 1 h. Again the mixture was cooled to −78° C. and anhydrous CuCl₂ (8.1 g, 60.90 mmol) was added in one portion, then it was warmed to room temperature and stirred overnight. After completion of reaction, the reaction mixture was quenched with water (50 mL) at 0° C., and it was subsequently extracted with hexane (3×100 mL). The organic phase was washed twice with water and the solvent was carefully removed to obtained crude DM-2F, which was purified by column separation (n-hexane) to afford a white crystalline solid (1.8 g, 72% yield).

¹H NMR (400 MHz, CDCl₃): δ 6.38 (d, J=3.2 Hz, 2H; H—C(2)), 6.03 (dd, J=3.3 Hz, 1.2 Hz, 2H; H—C(3)), 2.36 (d, J=1 Hz, 6H; H—C(5)); ¹³C NMR (101 MHz, CDCl₃): δ 151.3 (2 C(4)), 145.2 (2 C(1)), 107.2 (2 C(2)), 105.1 (2 C(3)), 13.6 (2 C(5)); HRMS (ESI): m/z calcd for C₁₀H₁₀O₂ (M+H)⁺: 163.0759, found: 163.0680.

4,4′-dimethyl-4H,4′H-1,1¹-bi(1,4-epoxynaphthalene) (DM-2CA)

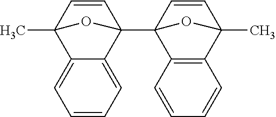

Finely powdered anhydrous CsF (0.937 g, 6.17 mmol) was added to a solution of 2-(Trimethylsilyl)phenyl trifluoromethanesulfonate 1 (0.278 mL, 1.35 mmol) and DM-2F (0.1 g, 0.617 mmol) in MeCN (2 mL), and the mixture was stirred at r.t. for 12 h. Then, the reaction mixture was filtered and solvent was removed under reduced pressure. The residue was purified by column chromatography (SiO₂; 9:1 hexane/EtOAc), affording DM-2CA as white crystalline solid (0.150 g, 77% yield).

¹H NMR (400 MHz, CDCl₃): δ 7.26 (dd, J=3.2 Hz, 4H), 7.12 (d, J=5.4 Hz, 2H), 7.06 (t, J=7.4 Hz, 2H), 7.00-6.91 (m, 4H), 2.03 (s, 6H); ¹³C NMR (101 MHz, CDCl₃) δ 152.7, 149.7, 147.1, 143.6, 125.0, 124.7, 121.1, 118.6, 90.4, 90.0, 15.3; HRMS (ESI): m/z calcd for C₂₂H₁₈O₂ (M+H)⁺: 315.1385, found: 315.1412.

4,4′-dimethyl-1,1′-binaphthalene (DM-2Nap)

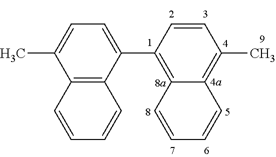

A solution of DM-2CA (0.02 g. 0.067 mmol) and anhydrous sodium iodide (0.048 g, 0.318 mmol) in 3 mL of dry acetonitrile was treated with trimethylsilyl chloride (0.04 mL, 0.318 mmol) at 0° C. under argon and stirred for 30 min. The reaction was quenched with the addition of 1 mL of 5% aqueous Na₂S₂O₃. The resulting mixture was then extracted with diethyl ether (50 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel with hexanes as the eluent to give DM-2Nap (10 mg, 56% yield) as white solid.

¹H NMR (400 MHz, CDCl₃): δ=8.10 (dd, J=8.5, 0.6 Hz, 2H; H—C(5)), 7.51 (ddd, J=8.3, 6.7, 1.4 Hz, 2H; H—C(6)), 7.43 (ddd, J=7.1, 6.3, 0.8 Hz, 4H; H—C(3 & 8)), 7.38 (d, J=7.1 Hz, 2H; H—C(2)), 7.28 (ddd, J=8.3, 6.7, 1.3 Hz, 2H; H—C(7)), 2.81 (d, J=0.9 Hz, 6H; H—C(9)); ¹³C NMR (126 MHz, CDCl₃) δ 137.0 (2 C(4)), 134.0 (2 C(1)), 133.0 (2 C(4a)), 132.6 (2 C(8a)), 127.6(2 C(2)), 127.3(2 C(8)), 126.2(2 C(3)), 125.6(2 C(6)), 125.5(2 C(7)), 124.2(2 C(5)), 19.6(2 C(9)); HRMS (FD): m/z calcd for C₂₂H₁₈: 282.1409 [M]⁺, found:282.1398.

4,4″-dimethyl-4H,4″H-1,1′:4′,1″-ter(1,4-epoxynaphthalene) (DM-3CA)

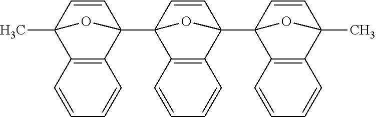

Finely powdered anhydrous CsF (0.665 g, 6.57 mmol) was added to a solution of 2-(Trimethylsilyl)phenyl trifluoromethanesulfonate 1 (0.296 mL, 1.44 mmol) and DM-3F (0.1 g, 0.438 mmol) in MeCN (3 mL), and the mixture was stirred at r.t. for 12 h. Then, the reaction mixture was filtered and solvent was removed under reduced pressure. The residue was purified by column chromatography (SiO₂; 9:1 hexane/EtOAc), affording DM-3CA as white solid (0.2 g, 80% yield).

¹H NMR (400 MHz, CDCl₃) δ 7.42 (dd, J=19.1, 6.6 Hz, 1H), 7.29-7.20 (m, 9H), 7.05-6.89 (m, 8H), 2.06 (d, J=2.4 Hz, 6H);¹³C NMR (101 MHz, CDCl₃) δ 152.5, 152.4, 150.2, 149.7, 149.6, 149.4, 149.3, 147.1, 146.9, 146.4, 144.5, 144.3, 144.2, 144.2, 143.8, 125.1, 125.0, 125.0, 125.0, 124.9, 124.8, 124.6, 124.7, 121.7, 118.6, 118.5, 15.4; HRMS (ESI): calcd for C₃₂H₂₄O₃ (M+H)⁺: 457.1804, found: 457.1804.

4,4″-dimethyl-1,1′:4′,1″-terbenzobenzene (DM-3Nap)

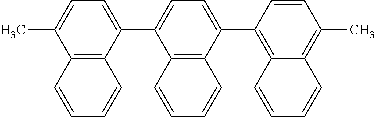

A solution of DM-3CA (0.036 g, 0.079 mmol) and anhydrous sodium iodide (0.059 g, 0.395 mmol) in 2 mL of dry acetonitrile was treated with trimethylsilyl chloride (0.03 mL, 0.236 mmol) at 0 ° C. under argon and stirred for 1 h. The reaction was quenched with the addition of 2 mL of 5% aqueous Na₂S₂O₃. The resulting mixture was then extracted with diethyl ether (30 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel with hexanes as the eluent to give DM-3Nap (20 mg, 63% yield) as a white solid.

¹H NMR (400 MHz, CDCl₃) δ ¹H NMR (500 MHz, Chloroform-d) δ 8.16-8.12 (m, 2H), 7.62 (ddd, J=8.5, 1.3, 0.7 Hz, 1H), 7.59 (d, J=1.8 Hz, 2H), 7.57-7.48 (m, 9H), 7.41 (ddd, J=8.3, 6.7, 1.3 Hz, 1H), 7.36 (ddd, J=8.3, 6.8, 1.2 Hz, 1H), 7.26-7.24 (m, 2H), 2.84 (d, J=0.6 Hz, 6H);¹³C NMR (126 MHz, CDCl₃) δ 138.47, 138.44, 136.93, 134.21, 134.19, 133.06, 133.04, 132.67, 132.65, 127.70, 127.69, 127.50, 127.35, 126.91, 126.26, 126.24, 125.72, 125.69, 125.67, 124.37, 124.33, 19.66; HRMS (FD): m/z calcd for C₃₂H₂₄: 408.1878, found: 408.1882.

5,5′″-dimethyl-2,2′:5′,2″:5″,2′″-quaterfuran (DM-4F)

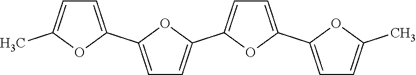

Pd(PPh₃)₄ (0.079 g, 5 mol %, 0.069 mmol) was added to a solution of 2,8-dibromobifuran (0.4 g, 1.37 mmol) and 2-methyl-5-stannyl furan (1.017 g, 2.74 mmol) in dry toluene (10 mL), and the reaction mixture was refluxed under nitrogen overnight and then cooled to room temperature. The mixture was quenched with water, extracted with hexane, dried (MgSO₄), and evaporated. Column chromatography on a silica column, using hexane as eluent gave DM-4F (0.260 g, 65% yield) as yellow colored solid.

¹H NMR (400 MHz, CDCl₃): δ 6.66 (d, J=3.5 Hz, 2H), 6.56 (d, J=3.5 Hz, 2H), 6.53 (d, J=3.3 Hz, 2H), 6.08 (dt, J=3.3, 1.0 Hz, 2H), 2.39 (d, J=1.0 Hz, 6H); ¹³C NMR (101 MHz, CDCl₃): δ 152.1, 107.6, 107.0, 106.5, 106.1, 76.7, 13.7; HRMS (ESI): m/z calcd for C₁₈H₁₄O₄: 294.0892, found: 294.0890.

4,4′″-dimethyl-4H,4′″H-1,1′:4′,1″:4″,1′″-quater(1,4-epoxynaphthalene) (DM-4CA)

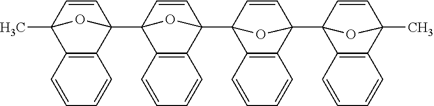

Finely powdered anhydrous CsF (1.029 g, 6.78 mmol) was added to a solution of 2-(Trimethylsilyl)phenyl trifluoromethanesulfonate 1 (0.296 mL, 1.44 mmol) and DM-4F (0.334 mL, 1.6 mmol) in MeCN (2 mL) and ethyl acetate (1 mL), and the mixture was stirred at r.t. for 16 h. Then, the reaction mixture was filtered and solvent was removed under reduced pressure. The residue was purified by column chromatography (9:1 hexane/EtOAc), affording DM-4CA as white solid (0.203 g, 88% yield).

¹H NMR (400 MHz, CDCl₃): δ 7.60-7.19 (m, 14 H), 7.11-6.86 (m, 10 H), 2.09 (d, J=1.7 Hz, 6H); ¹³C NMR (101 MHz, CDCl₃): δ 152.5, 152.4, 152.4, 150.0, 149.7, 149.7, 149.7, 149.2, 149.1, 147.1, 144.2, 144.0, 125.1, 125.1, 125.0, 124.9, 124.87, 124.8, 121.4, 118.6, 91.4, 90.3, 90.3, 90.0, 15.4; HRMS (ESI): m/z calcd for C₄₂H₃₀O₄: 598.2144, found: 598.2151.

4,4′″-dimethyl-1,1′:4′,1″:4″,1′″-quaternaphthalene (DM-4Nap)

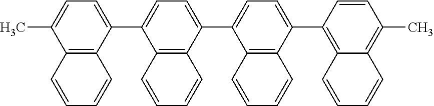

A solution of DM-4CA (0.05 g. 0.083 mmol) and anhydrous sodium iodide (0.116 g, 0.773 mmol) in 3 mL of dry acetonitrile was treated with trimethylsilyl chloride (0.071 mL, 0.560 mmol) at 0° C. under argon and stirred for 1 h. The reaction was quenched with the addition of 2 mL of 5% aqueous Na₂S₂O₃. The resulting mixture was then extracted with diethyl ether (20 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel with 5% hexane/ethyl acetate eluent to give DM-4Nap (27 mg, 61% yield) as a white solid.

¹H NMR (400 MHz, CDCl₃): δ 8.18 (dd, J=8.5, 4.2 Hz, 2H), 7.77-7.64 (m, 8H), 7.62-7.53 (m, 8H), 7.44-7.37 (m, 3H), 7.36-7.30 (m, 3H), 2.88 (s, 6H); ¹³C NMR (126 MHz, CDCl₃): 138.65, 138.64, 138.32, 138.28, 138.25, 136.88, 136.87, 136.85, 134.26, 134.24, 133.12, 133.11, 133.09, 133.02, 133.00, 132.99, 132.66, 132.65, 127.73, 127.71, 127.54, 127.35, 126.93, 126.27, 126.25, 125.84, 125.81, 125.79, 125.75, 125.71, 124.39, 124.35, 19.7; HRMS (FD): m/z calcd for C₄₂H₃₀: 534.2348, found: 534.2325

Synthesis of 5,5′″″-dimethyl-3′″,4″-diortyl-2,2′:5′,2″:5″,2′″:5′″,2″″:5″″, 2′″″-sexifuran (DM-6F-2C8)
5-methyl-2,2′-bifuran

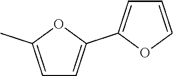

Pd(PPh₃)₄ (0.41 g, 0.36 mmol) was added to a solution of 2-methyl-5-iodofuran (1.5 g, 7.11 mmol) and 2-tributyltinfuran (2.27 mL, 7.11 mmol) in dry toluene (20 mL), and the reaction mixture was refluxed under nitrogen overnight and then cooled to room temperature. The mixture was quenched with water, extracted with hexane, dried (MgSO₄), and evaporated. Column chromatography on a silica column, using hexane as eluent gave 5-methyl-2,2′-bifuran (0.5 g, 74% yield) as colorless oil.

¹H NMR (400 MHz, CDCl₃): δ 7.40 (dd, J=1.8, 0.8 Hz, 1H), 6.50 (dd, J=3.4, 0.8 Hz, 1H), 6.46 (dd, J=3.4, 1.8 Hz, 2H), 6.09-6.03 (m, 1H), 2.37 (d, J=0.6 Hz, 3H); ¹³C NMR (101 MHz, CDCl₃): δ 151.7, 146.9, 145.0, 141.3, 111.3, 107.3, 106.0, 104.1, 13.6.

Tributyl(5′-methyl-[2,2′-bifuran]-5-yl)stannane

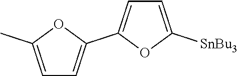

A solution of n-BuLi (4.81 mL, 1.6 M in hexanes, 7.70 mmol) was added dropwise to a solution of 5-methyl-2,2′-bifuran (0.88 g, 5.93 mmol) in dry tetrahydrofuran (20 mL) at −78° C. under N₂. The reaction mixture was allowed to reach room temperature and stirred for 1 h. The resulting mixture was cooled to −78° C., Bu₃SnCl (1.92 mL, 6.53 mmol) was added dropwise, and the reaction mixture was allowed to reach room temperature and stirred for 2 h. The mixture was quenched with water, extracted with hexane, dried (MgSO₄), and evaporated. Hash chromatography on basified (NEt₃) silica, using hexane as eluent gave tributyl(5′-methyl[2,2′-bifuran]-5-yl)stannane (1.1 g, 42% yield) as a pale yellow oil.

¹H NMR (400 MHz, Chloroform-d): δ 6.59 (d, J=3.2 Hz, 1H), 6.52 (d, J=3.2 Hz, 1H), 6.43 (d, J=3.2 Hz, 1H), 6.04 (dd, J=3.2, 1.0 Hz, 1H), 2.37-2.36 (d, J=1.0 Hz, 3H), 1.64-1.58 (m, 6H), 1.41-1.35 (m, 6H), 1.15-1.08 (m, 6H), 0.91 (d, J=7.3 Hz, 9H); ¹³C NMR (126 MHz, Chloroform-d) δ 160.1, 151.3, 145.9, 122.9, 107.2, 105.6, 104.1, 28.9, 27.1, 13.7, 13.6, 13.6, 10.2.

5,5′″″-dimethyl-3′″,4″-dioctyl-2,2′:5′,2″:5″:2′″:5′″,2″″:5″″,2′″″-sexifuran (DM-6F-2C8)

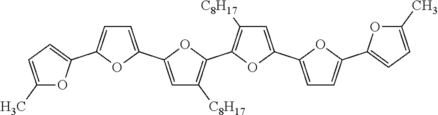

Pd(PPh3)4 (0.034 g, 0.029 mmol) was added to a solution of 5,5′-dibromo-3,3′-dioctyl-2,2′-bifuran (0.3 g, 0.580 mmol) and tributyl(5′-methyl-[2,2′-bifuran]-5-yl)stannane (0.508 g, 1.16 mmol) in dry toluene (5 mL), and the reaction mixture was refluxed under nitrogen overnight and then cooled to room temperature. The mixture was quenched with water, extracted with hexane, dried (MgSO₄), and evaporated. Column chromatography on a silica column, using hexane as eluent gave DM-6F-2C₈ (0.150 g, 40% yield) as yellow solid.

¹H NMR (400 MHz, CDCl₃): δ 6.62 (s, 2H), 6.57 (d, J=3.3 Hz, 4H), 6.54-6.49 (m, 2H), 6.08 (dd, J=3.2, 1.0 Hz, 2H), 2.86-2.74 (m, 4H), 2.39 (s, 6H), 1.75-1.67 (m, 4H), 1.45-1.27 (m, 20H), 0.90 (d, J=6.7 Hz, 6H);¹³C NMR (101 MHz, CDCl₃) δ 152.0, 145.3, 144.7, 141.7, 124.7, 108.8, 107.6, 106.7, 106.4, 106.1, 31.9, 31.9, 30.3, 29.6, 29.5, 29.3, 25.4, 22.7, 14.1, 13.7; HRMS (ESI): m/z calcd for C₄₂H₅₀O₆: 650.3607, found: 650.3623.

Synthesis of 4,4′″″-dimethyl-2′″,3″-dioctyl-4H,4′″″H-1,1′:4′,1″:4″,1′″:4′″,1″″:4″″,1′″″-sexi(1,4-epoxynaphthalene) (DM-6CA-2C8)

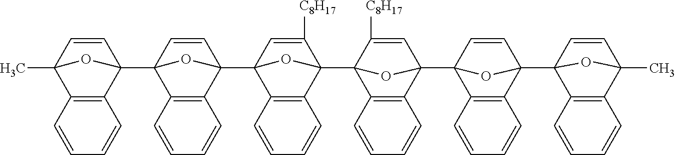

Finely powdered anhydrous CsF (0.350 g, 2.31 mmol) was added to a solution of 2-(Trimethylsilyl)phenyl trifluoromethanesulfonate 1 (0.113 mL, 0.553 mmol) and DM-6F-2C₈ (0.334 mL, 0.077 mmol) in MeCN (2 mL) and ethyl acetate (2 mL), and the mixture was stirred at r.t. for 12 h. Then, the reaction mixture was filtered and solvent was removed under reduced pressure. The residue was purified by column chromatography (7:3 hexane/EtOAc), affording DM-6CA-C8 as white glassy solid (0.07 g, 82% yield).

¹H NMR (400 MHz, CDCl₃): δ 7.43-7.30 (m, 10H), 7.27-7.19 (m, 4H), 7.11-6.83 (m, 20H), 2.87-2.29 (m, 4H), 2.11 (s, 6H), 1.57-1.43 (m, 4H), 1.31-1.14 (m, 20H), 0.87 (t, J=5.9 Hz, 6H); ¹³C NMR (101 MHz, Chloroform-d) δ 152.5, 152.4, 150.5, 149.7, 149.4, 149.3, 146.9, 144.8, 144.2, 144.0, 125.1, 125.0, 124.9, 124.8, 124.7, 124.6, 122.1, 122.0, 121.9, 121.6, 121.5, 118.6, 118.5, 91.6, 90.5, 90.3, 90.2, 90.0, 31.8, 31.7, 29.4, 29.3, 29.2, 29.1, 26.8, 22.6, 15.4, 15.3, 14.1; HRMS (ESI): m/z calcd for C₇₈H₇₄O₆: 1106.5485, found: 1106.5525.

### DM-6F-Ar (DM-6Nap-2C8)

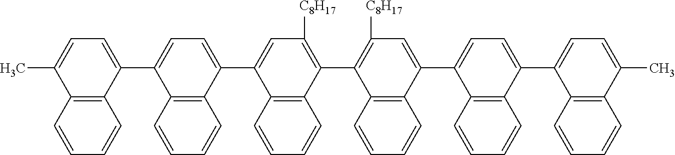

A solution of DM-6CA (0.08 g. 0.07 mmol) and anhydrous sodium iodide (0.151 g, 1.0 mmol) in 2 mL of dry acetonitrile and 2 mL of ethyl acetate was treated with trimethylsilyl chloride (0.1 mL, 0.722 mmol) at 0° C. under argon and stirred for 1 h. The reaction was quenched with the addition of 3 mL of 5% aqueous Na₂S₂O₃. The resulting mixture was then extracted with diethyl ether (20 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel with hexanes as the eluent to give DM-6Nap (38 mg, 52% yield) as a white solid.

¹H NMR (500 MHz, Chloroform-d) δ 8.20-8.10 (m, 4H), 7.83-7.77 (m, 2H), 7.72-7.63 (m, 8H), 7.60-7.52 (m, 12H), 7.40-7.29 (m, 12H), 2.87-2.85 (m, 6H), 2.85-2.77 (m, 4H), 1.79-1.68 (m, 5H), 1.29 (m, 6H), 1.20 (d, J=16.4 Hz, 15H), 0.91-0.86 (t, J=7.1 Hz, 6H); ¹³C NMR (126 MHz, Chloroform-d) δ 134.27, 134.26, 134.24, 133.19, 133.07, 133.05, 133.01, 127.76, 127.74, 127.72, 127.59, 127.55, 126.98, 126.93, 126.28, 126.26, 125.90, 125.87, 125.84, 125.82, 125.79, 125.75, 125.71, 124.46, 124.40, 124.35, 29.72, 29.39, 22.72, 22.66, 19.68, 14.14.

4,4′-dimethyl-4H,4′H-1,1′-bi(1,4-epoxytriphenylene) (3)

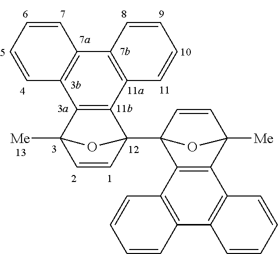 [SMILES: CC12C=CC(C34C=CC(C)(O3)c3c4c4ccccc4c4ccccc34)(O1)c1c2c2ccccc2c2ccccc12]

Finely powdered anhydrous CsF (0.468 g, 3.08 mmol) was added to a solution of phenanthrene precursor (10-trimethylsilylphenanthryl 9-trifluoromethanesulfonate) 2 (0.246 g, 0.617 mmol) and DM-2F (0.05 g, 0.308 mmol) in MeCN (2 mL) and ethyl acetate (2 mL), and the mixture was stirred at r.t. for 12 h. After completion of the reaction, mixture was filtered and solvent was removed under reduced pressure. The residue was purified by column chromatography (7:2 hexane/EtOAc), affording phenanthrene cyclo-adduct 3 as a white solid (0.07 g, 44% yield).

¹H NMR (500 MHz, Chloroform-d) δ 8.70 (J=8.3, 1.4, 0.6 Hz, 2H; H—C(8)), 8.46 (dd, J=16.9, 8.2 Hz, 4H; H—C(9 & 12)), 8.37 (ddd, J=8.3, 1.4, 0.6 Hz, 2H; H—C(5)), 7.68 (ddd, J=8.3, 7.0, 1.4 Hz, 2H); H—C(6), 7.63 (ddd, J=8.3, 7.0, 1.4 Hz, 2H; H—C(1)), 7.59 (d, J=5.1 Hz, 2H; H—C(2)), 7.22 (ddd, J=8.3, 7.0, 1.2 Hz, 2H; H—C(10)), 7.18 (d, J=5.1 Hz, 2H; H—C(3)), 6.91 (ddd, J=8.3, 6.9, 1.2 Hz, 2H; H—C(11)), 2.63 (s, 6H; H—C(13)); ¹³C NMR (126 MHz, Chloroform-d) δ 149.3 (2 C(12b)), 147.9 (2 C(4a)), 147.8 (2 C(3)), 147.4 (2 C(2)), 129.8(2 C(8a)), 128.9 (2 C(8b)), 127.6 (2 C(4a)), 127.5 (2 C(12a)), 126.3 (2 C(12)), 126.2 (2 C(11)), 126.1 (2 C(6)), 125.6 (2 C(7)), 125.5 (2 C(10)), 123.8 (2 C(8)), 123.2 (2 C(5)), 122.4 (2 C(9)), 93.1 (2 C(1)), 92.2 (2 C(4)), 19.7 (2 C(13)); HRMS (ESI): m/z calcd for C₃₈H₂₆O₂ (M+H)⁺: 515.2011, found: 515.2024.

4,4′-dimethyl-1,1′-bitriphenylene (4)

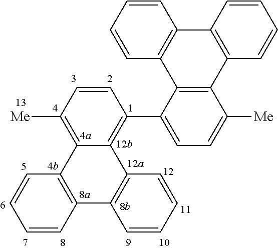 [SMILES: Cc1ccc(-c2ccc(C)c3c4ccccc4c4ccccc4c23)c2c3ccccc3c3ccccc3c12]

A solution of phenanthrene cyclo-adduct 3 (0.02 g. 0.039 mmol) and anhydrous sodium iodide (0.06 g, 0.388 mmol) in dry acetonitrile (2 mL) and dry dichloromethane (2 mL) was treated with trimethylsilyl chloride (0.05 mL, 0.388 mmol) at 0° C. under argon and stirred for 6 h. The reaction was quenched with the addition of 2 mL of 5% aqueous Na₂S₂O₃. The resulting mixture was then extracted with diethyl ether (20 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel (9:1 hexanes/EtOAc) to give 4,4′-dimethyl-1,1′-bitriphenylene 4 (12 mg; 64% yield) as a white solid.

¹H NMR (500 MHz, Chloroform-d) δ 8.62 (dd, J=8.1, 1.4 Hz, 2H; H—C(8)), 8.55 (dd, J=8.1, 1.3 Hz, 2H; H—C(9)), 8.50 (dd, J=8.2, 1.3 Hz, 2H; H—C(5)), 8.28 (dd, J=8.6, 0.8 Hz, 2H; H—C(12)), 7.67 (ddd, J=8.2, 7.1, 1.3 Hz, 2H; H—C(7)), 7.60 (ddd, J=8.4, 7.1, 1.4 Hz, 2H; H—C(6)), 7.45 (ddd, J=8.2, 7.0, 1.2 Hz, 2H; H—C(10)), 7.27 (d, J=0.7 Hz, 2H; H—C(3)), 7.08 (d, J=7.5 Hz, 4H; H—C(2)), 7.06 (ddd, J=8.4, 7.0, 1.4 Hz, 4H; H—C(11)), 3.02 (s, 6H; H—C(13)); ¹³C NMR (126 MHz, Chloroform-d) δ¹³C NMR (126 MHz, Chloroform-d) δ 140.0 (2 C(4)), 133.3 (2 C(4a)), 133.3 (2 C(1)), 131.2 (2 C(8a)), 131.1(2 C(8b)), 131.0 (2 C(2)), 130.9 (2 C(4b)), 130.7 (2 C(12a)), 130.7 (2 C(3)), 129.6 (2 C(12b)), 129.0 (2 C(12)), 128.5 (2 C(5)), 126.9(2 C(7)), 126.6 (2 C(10)), 126.2 (2 C(11)), 125.9 (2 C(6)), 123.5(2 C(8)), 123.2 (2 C(9)), 25.5(2 C(13)). HRMS (FD): m/z calcd for C₃₈H₂₆: 482.2035, found: 482.2030.

[1,1′-binaphthalene]-4,4′-diol

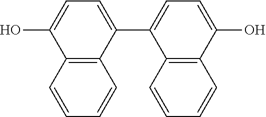 [SMILES: Oc1ccc(-c2ccc(O)c3ccccc23)c2ccccc12]

A solution of DM-2CA (0.05 g, 0.175 mmol) and anhydrous sodium iodide (0.078 g, 0.525 mmol) in 2 mL of dry acetonitrile was treated with trimethylsilyl chloride (0.05 mL, 0.384 mmol) at 0° C. under argon and stirred for 1 h. The reaction was quenched with the addition of 2 mL of 5% aqueous Na₂S₂O₃. The resulting mixture was then extracted with diethyl ether (20 mL). The organic layer was washed with 5% aqueous Na₂S₂O₃ (2 mL) and brine (5 mL) and dried over anhydrous MgSO₄ and the solvent was evaporated under reduced pressure. Then the residue was purified by flash column chromatography on silica gel with 20% hexane/ethyl acetate eluent to give 1,1′-binaphthalene-4,4′-diol (35 mg, 70% yield) as a white solid.

¹H NMR (400 MHz, DMSO-d₆) δ 10.24 (s, 2H), 8.23 (ddd, J=8.4, 1.4, 0.7 Hz, 2H), 7.42 (ddd, J=8.3, 6.7, 1.3 Hz, 2H), 7.28 (ddd, J=8.2, 6.7, 1.4 Hz, 2H), 7.23 (d, J=7.7 Hz, 2H), 7.18-7.15 (m, 2H), 6.98 (d, J=7.7 Hz, 2H); ¹³C NMR (101 MHz, DMSO-d6) 153.2, 134.2, 129.2, 128.9, 126.5, 126.3, 125.0, 124.8, 122.8, 108.2; HRMS (ESI): m/z calcd for C₂₉H₁₄O₂: 286.0994, found: 286.1010.

2,4-dimethyl-7-(5′-methyl-[2,2′-bifuran]-5-yl)isoindoline-1,3-dione

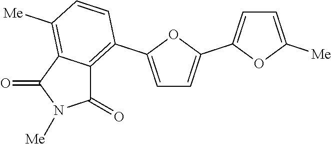 [SMILES: Cc1ccc(-c2ccc(-c3ccc(C)c4c3C(=O)N(C)C4=O)o2)o1]

DM-3F (44 mg, 0.192 mmol), N-methyl maleimide 5 (32 mg, 0.289 mmol) and p-toluene sulfonyl anhydride (73 mg, 0.384 mmol) were added to ethyl acetate (0.5 ml) and stirred for 12 h at 60° C. After cooled to room temperature, the reaction was stopped by adding sat. aq. NaHCO₃ (1 mL). The products were extracted with EtOAc (10 mL), and the combined organic extracts were washed with brine, dried (Na₂SO₄), and concentrated in vacuo. The residue was purified by silica-gel flash column chromatography (7:3 hexane/ethyl acetate) to give 2,4-dimethyl-7-(5′-methyl-[2,2′-bifuran]-5-yl)isoindoline-1,3-dione (30 mg, remaining starting material) as yellow solid.

¹H NMR (400 MHz, Chloroform-d) δ 8.11 (d, J=8.3 Hz, 1H), 8.03 (d, J=3.7 Hz, 1H), 7.46 (dd, J=8.3, 0.8 Hz, 1H), 6.66 (d, J=3.7 Hz, 1H), 6.60 (d, J=3.3 Hz, 1H), 6.10 (dd, J=3.3, 1.0 Hz, 1H), 3.19 (s, 3H), 2.72 (s, 3H), 2.40 (d, J=0.5 Hz, 3H);); ¹³C NMR (200 MHz, CDCl₃) δ 152.6, 147.6, 147.0, 144.6, 136.5, 136.1, 130.8, 129.8, 126.6, 124.4, 116.3, 107.7, 107.3, 106.9, 23.8, 17.7, 13.7; HRMS (ESI): m/z calcd for C₁₉H₁₅NO₄ (M+H)⁺: 322.1079, found: 322.1034.

2,4-dimethyl-7-(4′-methyl-[1,1′-binaphthalen]-4-yl)isoindoline-1,3-dione (8)

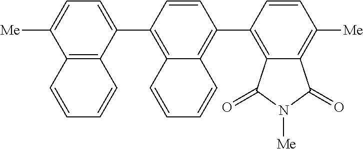 [SMILES: Cc1ccc(-c2ccc(-c3ccc(C)c4ccccc34)c3ccccc23)c2c1C(=O)N(C)C2=O]

In a vial, finely powdered anhydrous CsF (109 mg, 0.716 mmol) was added to a solution of 2-(Trimethylsilyl)phenyl trifluoromethanesulfonate 1 (0.03 mL, 0.150 mmol) and 2,4-dimethyl-7-(5′-methyl-[2,2′-bifuran]-5-yl)isoindoline-1,3-dione (0.024 g, 0.0716 mmol) in acetonitrile (0.5 mL) and ethyl acetate (0.5 mL), and the mixture was stirred at r.t. for 12 h. After completion of the reaction, mixture was filtered and solvent was removed under reduced pressure. The residue was dried under high vacuo, affording cyclo-adduct as a white solid (30 mg, 86% yield). HRMS (ESI): m/z calcd for C₃₁H₂₃N₂O₅ (M+Na)⁺: 496.1525, found: 496.1501.

Further, the solution of crude cyclo-adduct (20 mg, 0.042 mmol) and anhydrous sodium iodide (29 mg, 0.196 mmol) in acetonitrile (05 mL) and ethyl acetate (0.5 mL) was treated with trimethylsilyl chloride (0.018 mL, 0.139 mmol) at 0° C. under argon and stirred for 6 h. The reaction was quenched with the addition of 2 mL of 5% aqueous Na₂S₂O₃. The resulting mixture was then extracted with diethyl ether (20 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel (8:2 hexanes/EtOAc) to give oligoarene 8 (12 mg, 65% yield) as a white solid.

¹H NMR (500 MHz, Chloroform-d) δ 8.14-8.09 (m, 1H), 7.68 (d, J=7.8 Hz, 1H), 7.61-7.56 (m, 3H), 7.55-7.51 (m, 3H), 7.50-7.41 (m, 3H), 7.39-7.33 (m, 2H), 7.32-7.26 (m, 1H), 3.10 (d, J=8.2 Hz, 3H), 2.83 (d, J=5.6 Hz, 6H);); ¹³C NMR (126 MHz, Chloroform-d) δ 169.09, 167.72, 167.65, 139.36, 139.29, 137.24, 137.22, 136.71, 136.66, 136.54, 136.51, 136.04, 135.99, 134.59, 134.39, 134.28, 134.20, 133.06, 133.00, 132.90, 132.89, 132.58, 132.57, 131.85, 131.79, 129.95, 129.92, 129.52, 129.47, 127.85, 127.55, 127.41, 127.25, 127.17, 127.13, 126.63, 126.54, 126.30, 126.06, 126.02, 125.98, 125.91, 125.87, 125.80, 125.59, 125.56, 125.47, 125.43, 124.35, 124.15, 29.69, 23.67, 23.65, 19.63, 19.60, 17.68; HRMS (ESI): m/z calcd for C₂₅H₂₀N₂O₅(M+H)⁺: 442.1807, found: 442.1805.

4-(5-(2,7-dimethyl-1,3-dioxoisoindolin-4-yl)furan-2-yl)-2-ethyl-7-methylisoindoline-1,3-dione

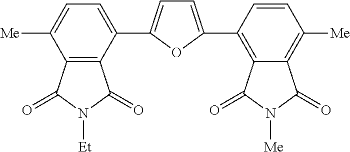 [SMILES: CCN1C(=O)c2c(C)ccc(-c3ccc(-c4ccc(C)c5c4C(=O)N(C)C5=O)o3)c2C1=O]

2,4-dimethyl-7-(5′-methyl-[2,2′-bifuran]-5-yl)isoindoline-1,3-dione (20 mg, 0.06 mmol), N-ethyl maleimide 6 (8 mg, 0.06 mmol) and p-toluene sulfonyl anhydride (17 mg, 0.09 mmol) were added to ethyl acetate (0.5 ml) and stirred for 12 h at 60° C. After cooled to room temperature, the reaction was quenched by adding sat. aq. NaHCO₃ (1 mL). The products were extracted with EtOAc (10 mL), and the combined organic extracts were washed with brine, dried over Na₂SO₄, and concentrated in vacuo. The residue was purified by silica-gel flash column chromatography (7:3 hexane/ethyl acetate) to give 4-(5-(2,7-dimethyl-1,3-dioxoisoindolin-4-yl)furan-2-yl)-2-ethyl-7-methylisoindoline-1,3-dione (15 mg, 58% yield) as yellow solid.

¹-11 NMR (400 MHz, Chloroform-d) δ 8.17 (d, J=8.2 Hz, 2H), 8.00 (s, 2H), 7.51 (dd, J=8.2, 0.8 Hz, 2H), 3.77 (q, J=7.2 Hz, 2H), 3.20 (s, 3H), 2.74 (s, 6H), 1.29 (t, J=7.3 Hz, 3H); ¹³C NMR (101 MHz, Chloroform-d) δ 168.7, 168.4, 167.9, 167.6, 149.3, 149.3, 136.9, 136.9, 136.6, 136.6, 131.3, 130.0, 130.0, 126.4, 126.3, 125.5, 125.5, 116.5, 32.8, 23.9, 17.8, 17.8, 13.9; HRMS (ESI): m/z calcd for C₃₁H₂₃NO₂ (M+H)⁺: 429.1450, found: 429.1409.

4-(4-(2,7-dimethyl-1,3-dioxoisoindolin-4-yl)naphthalen-1-yl)-2-ethyl-7-methylisoindoline-1,3-dione (9)

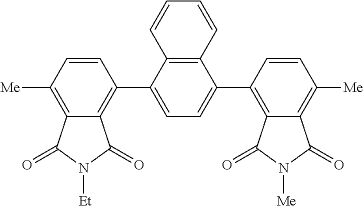 [SMILES: CCN1C(=O)c2c(C)ccc(-c3ccc(-c4ccc(C)c5c4C(=O)N(C)C5=O)c4ccccc34)c2C1=O]

In a vial, finely powdered anhydrous CsF (30 mg, 0.192 mmol) was added to a solution of 2-(Trimethylsilyl)phenyl trifluoromethanesulfonate 1 (0.011 mL, 0.053 mol) and 4-(5-(2,7-dimethyl-1,3-dioxoisoindolin-4-yl)furan-2-yl)-2-ethyl-7-methylisoindoline-1,3-dione (0.02 g, 0.048 mmol) in acetonitrile (0.5 mL) and ethyl acetate (0.5 mL), and the mixture was stirred at r.t. for 12 h. After completion of the reaction, mixture was filtered and solvent was removed under reduced pressure. The residue was dried under high vacuo, affording cyclo-adduct as a white solid (15 mg, 64% yield), HRMS (FD): m/z calcd for C₃₁H₂₄N₂O₅(M+Na)⁺: 527.1583, found: 527.1572 Further, the solution of crude cyclo adduct (15 mg, 0.03 mmol) and anhydrous sodium iodide (0.01 g, 0.066 mmol) in dry acetonitrile (2 mL) and dry dichloromethane (1 mL) was treated with trimethylsilyl chloride (8 μL, 0.06 mmol) at 0° C. under argon and stirred for 6 h. The reaction was quenched with the addition of 2 mL of 5% aqueous Na₂S₂O₃. The resulting mixture was then extracted with diethyl ether (10 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel (8:2 hexanes/EtOAc) to give deoxygenated product 9 (7 mg, 48% yield) as a white solid.

7,7′-(furan-2,5-diyl)bis(2,4-dimethylisoindoline-1,3-dione)

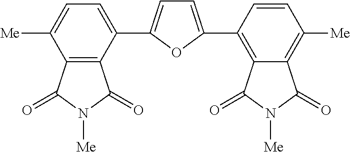 [SMILES: Cc1ccc(-c2ccc(-c3ccc(C)c4c3C(=O)N(C)C4=O)o2)c2c1C(=O)N(C)C2=O]

DM-3F (44 mg, 0.192 mmol), N-methyl maleimide 5 (43 mg, 0.384 mmol) and p-toluene sulfonyl anhydride (73 mg, 0.384 mmol) were added to ethyl acetate (1 ml) and stirred for 8 h at 60° C. After cooled to room temperature, the reaction was quenched by adding sat. aq. NaHCO₃ (1 mL). The products were extracted with EtOAc (15 mL), and the combined organic extracts were washed with brine, dried over Na₂SO₄, and concentrated in vacuo. The residue was purified by silica-gel flash column chromatography (5:5 hexane/ethyl acetate) to give 7,7′-(furan-2,5-diyl)bis(2,4-dimethylisoindoline-1,3-dione) (50 mg, 61% yield) as yellow solid.

¹H NMR (400 MHz, Chloroform-d) δ 8.14 (dd, J=8.3, 0.5 Hz, 2H), 7.98 (s, 2H), 7.50 (d, J=0.7 Hz, 1H), 7.48 (d, J=0.7 Hz, 1H), 3.18 (s, 6H), 2.72 (s, 6H); ¹³C NMR (101 MHz, Chloroform-d) δ 168.6, 167.8, 149.3, 136.9, 136.6, 131.3, 130.0, 126.3, 125.4, 116.5, 23.8, 17.8; HRMS (ESI): m/z calcd for C₂₄H₁₈N₂O₅ (M+Na)⁺: 414.1113, found: 437.1100.

7,7′-(naphthalene-1,4-diyl)bis(2,4-dimethylisoindoline-1,3-dione) (7)

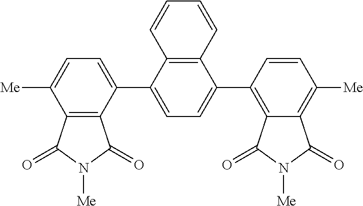 [SMILES: Cc1ccc(-c2ccc(-c3ccc(C)c4c3C(=O)N(C)C4=O)c3ccccc23)c2c1C(=O)N(C)C2=O]

In a vial, finely powdered anhydrous CsF (30 mg, 0.192 mmol) was added to a solution of 2-(Trimethylsilyl)phenyl trifluoromethanesulfonate 1 (0.011 mL, 0.053 mmol) and 7,7′-(furan-2,5-diyl)bis(2,4-dimethylisoindoline-1,3-dione) (0.02 g, 0.048 mmol) in acetonitrile (0.5 mL) and ethyl acetate (0.5 mL), and the mixture was stirred at r.t. for 12 h. After completion of the reaction, mixture was filtered and solvent was removed under reduced pressure. The residue was dried under high vacuo, affording cyclo-adduct as a white solid (15 mg, 64% yield), HRMS (FD): m/z calcd for C₃₀H₂₂N₂O₅: 490.1607, found: 490.1624.

Further, the solution of crude cyclo adduct (15 mg, 0.03 mmol) and anhydrous sodium iodide (0.01 g, 0.066 mmol) in dry acetonitrile (1 mL) and dry dichloromethane (2 mL) was treated with trimethylsilyl chloride (8 μL, 0.06 mmol) at 0° C. under argon and stirred for 6 h. The reaction was quenched with the addition of 2 mL of 5% aqueous Na₂S₂O₃. The resulting mixture was then extracted with diethyl ether (10 mL). The organic layer was washed with 5% aqueous Na₂S₂O₃ (2 mL) and brine (5 mL) and dried over anhydrous MgSO₄ and the solvent was evaporated under reduced pressure. The residue thus obtained was purified by flash column chromatography on silica gel (8:2 hexanes/EtOAc) to give deoxygenated product 7 (7 mg, 48% yield) as a white solid.

¹H NMR (400 MHz, Chloroform-d) δ 7.65 (d, J=7.8 Hz, 2H; H—C(8)), 7.59-7.54 (m, 4H), 7.48 (s, 2H), 7.37 (dd, J=6.5, 3.3 Hz, 2H), 3.07 (s, 6H), 2.82 (s, 6H); ¹³C NMR (126 MHz, Chloroform-d) δ 169.05, 167.71, 137.32, 136.78, 136.31, 136.15, 136.06, 135.88, 135.24, 131.83, 129.84, 129.41, 126.27, 126.18, 126.14, 126.08, 125.78, 125.71, 29.71, 23.64, 17.68.

HRMS (ESI): m/z calcd for C₃₀H₂₂N₂O₄ (M+Na)⁺: 497.1477, found: 497.1484.

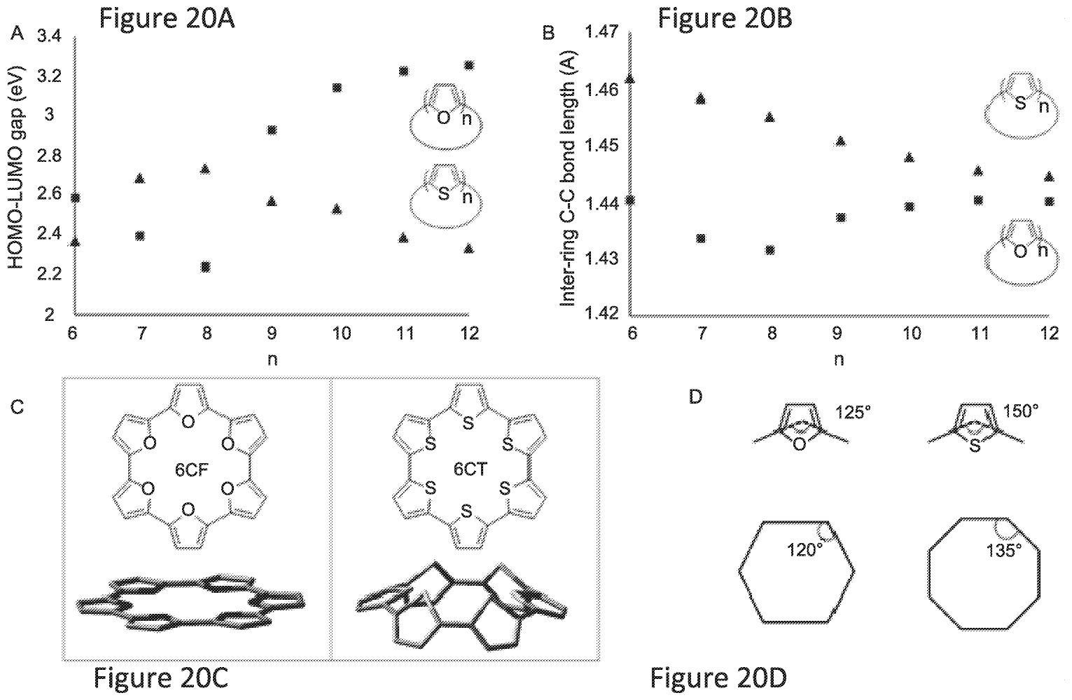

The physical properties of linear oligomers are influenced by undesired chain-end effects. In this respect, corresponding fully π-conjugated macrocycles represent model systems that combine the ‘infinite’ defect-free π-conjugated chain of an idealized polymer with the advantage of a structurally well-defined oligomer, while excluding perturbing end-effects. This renders them suitable candidates for various applications in organic and molecular electronics and for the study of host-guest interactions, aggregation, and self-assembly on surfaces. While macrocyclic oligothiophenes were extensively explored, macrocyclic oligofurans remain unknown. Computational studies demonstrate that these materials exhibit highly attractive properties, namely:
(i) Planarity: For oligothiophenes, relatively small macrocyclic systems were calculated to be non-planar, mainly due to strain. In furan-based systems, however, the exo angle is ˜8° larger than in thiophene (133° and 125° respectively, FIG. 20) and, as a result, smaller planar macrocyclic systems were calculated to a minimal structure. For example, the 8-membered cyclic oligofuran system is calculated to be completely planar, in contrast to its cyclic oligothiophene analogue. In addition, alkyl groups can be integrated into the polymer to increase solubility and form different types of assemblies, without any significant perturbation of planarity. Such planarity is crucial for π-conjugation, making macrocyclic oligofurans attractive candidates for organic electronic materials.
(ii) Quinoid character: Oligofurans display a significantly stronger quinoid character compared with oligothiophenes, as evident from both DFT calculations and X-ray structure. The results demonstrate that this trend is even more pronounced for macrocyclic oligofurans. For example, 8CF shows a strong quinoid character, with the inter-ring C—C bond distance being 1.431 Å compared with 1.455 Å for 8CT (FIG. 20B).
(iii) HOMO-LUMO gap (HLG): In general, the HLG of linear oligofurans is ca. 0.3 eV higher than that of the corresponding oligothiophenes, with the HLG decreasing with oligomer length to eventually converge to a value of 2.5 eV. Macrocyclic oligofurans differ markedly from this trend, with the HLG decreasing to a value of 2.24 eV for 8CF, which is significantly lower than that of either linear 8F (2.8 eV), polyfuran (2.41 eV) or macrocylic oligothiophenes (FIG. 20A).

Overall, the computational results reveal that macrocyclic oligofurans, in particular 6CF-8CF, are suitable electronic materials, owing to their planarity, low strain energy, low HOMO-LUMO gap, and strong quinoid character.

1.-79. (canceled)

80. A method for the synthesis of a π-conjugated system, the method comprising reacting an oligofuran with at least one aryne or heteroaryne to convert each furan moiety in said oligofuran to a cycloadduct and reacting said cycloadduct to yield the n-conjugated system.

81. The method according to claim 80, wherein the cycloadduct is an extended cycloadduct prepared by reacting the cycloadduct with at least one diene, prior to conversion to the π-conjugated system.

82. The method according to claim 80, wherein the cycloadduct is an oxo- or syn-cycloadduct, such that each cycloadduct is in the form of an oxo-cyclocadduct or a syn-cycloadduct.

83. The method according to claim 80, wherein the aryne or heteroaryne is a multicyclic-aryne or multicyclic-heteroaryne.

84. The method according to claim 80, wherein the n-conjugated system is selected amongst oligoarenes, oligoacenes, graphene segments and carbon nanobelts.

85. The method according to claim 80, wherein the oligofuran is selected amongst linear oligofurans and cyclic oligofurans.

86. The method according to claim 80, wherein the oligofuran is of the structure: 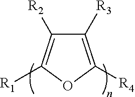
wherein
n is an integer defining the number of furan moieties that are bonded to each other;
each of R₁, R₂, R₃ and R₄ is independently selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen,
wherein each of R′, R″ and R′″ is the same or different and is selected from hydrogen; C₁-C₁₀alkyl; C₂-C₁₀alkenyl; C₂-C₁₀alkynyl; and C₆-C₁₀aryl;
or
R₁ and R₂, or R₂ and R₃, or R₃ and R₄, together with the carbon atoms to which they are bonded form a 4-8-membered ring comprising between 0 and 3 double bonds, and/or between 0 and 3 heteroatoms selected from O, N, and S;
the 4-8 membered ring system being optionally substituted by at least one group selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; and C₆-C₁₀aryl;
wherein at least one of R₁, R₂, R₃ and R₄ is a furan ring moiety of the oligofuran chain.

87. The method according to claim 86, wherein at least one of R₁ and R₄ is a furan ring moiety.

88. The method according to claim 86, wherein one of R₁ and R₄ is a furan ring moiety and the other of R₁ and R₄ is selected from hydrogen; C₁-C₁₀alkyl; —CN; —CO₂H; (C₁-C₁₀alkyl)₃Sn—; and a halogen.

89. The method according to claim 86, wherein one of R₁ and R₄ is a furan ring moiety and the other of R₁ and R₄ is selected from hydrogen; an alkyl stannyl and a halogen.

90. The method according to claim 80, wherein the oligofuran is linear, each end-of-chain furan having an a-carbon substituted by a group selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; an alkyl stannyl; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; and C₆-C₁₀aryl.

91. The method according to claim 86, wherein the cyclic oligofuran is of the structure: 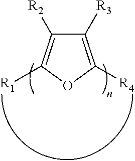
wherein each of n, R₁, R₂, R₃ and R₄ are as defined in claim 86.

92. The method according to claim 80, wherein the π-conjugated system is of the structure: 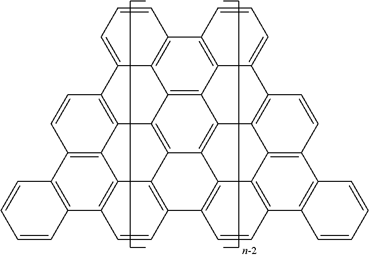
wherein
each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and
n is an integer between 2 and 100;
or 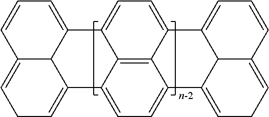
wherein
each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and
n is an integer between 2 and 100;
or 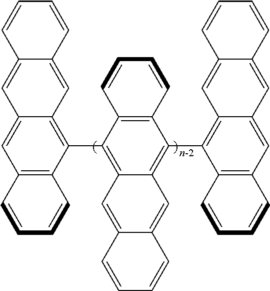
wherein
each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and
n is an integer between 2 and 100.

93. A compound having the structure: 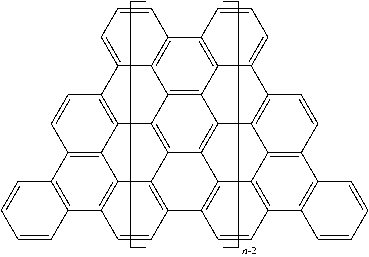
wherein
each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and
n is an integer between 2 and 100;
or a compound of the structure: 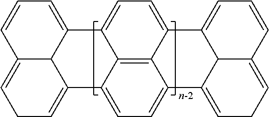
wherein
each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and
n is an integer between 2 and 100;
or a compound of the structure: 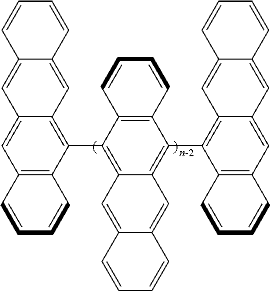
wherein
each peripheral carbon atom is substituted with a group or an atom selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O and S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ is the same or different and is selected from hydrogen; C₁-C₁₀alkyl; C₂-C₁₀alkenyl; C₂-C₁₀alkynyl; C₆-C₁₀aryl; and
n is an integer between 3 and 100.

94. A method for the synthesis of a cyclic oligofuran, the method comprising:
reacting an alkyne-substituted oligofuran in the presence of a metal complex to afford an alkyne-metal macrocycle,
treating said macrocycle to obtain a di-acetylene macrocycle and
oxidizing said di-acetylene macrocycle to afford the cyclic oligofuran.

95. A method for the synthesis of a cyclic oligofuran, the method comprising:
reacting furan with succinyl chloride to afford a linear furan dimer, wherein each furan is separated by a succinyl moiety,
optionally repeating the reaction to afford higher homologs of the furan dimer,
reacting the furan dimer or higher homolog thereof with succinyl chloride under conditions permitting cyclization into a cyclic macrocycle, and
transforming each succinyl moiety of the cyclic macrocycle to a furan, thereby providing the cyclic oligofuran.

96. A method for the synthesis of a cyclic oligofuran, the method comprising:
reacting a linear oligofuran with succinyl chloride to afford a linear oligofuran dimer, wherein each oligofuran is separated by a succinyl moiety,
optionally repeating the reaction to afford higher homologs of the dimer,
reacting the dimer or higher homolog thereof with succinyl chloride under conditions permitting cyclization into a cyclic macrocycle, and
transforming each succinyl moiety of the cyclic macrocycle to a furan, thereby providing the cyclic oligofuran.

97. A cyclic oligofuran comprising between 4 and 20 furan ring moieties, each furan ring moiety being covalently associated to another via the furan a-carbon atoms.

98. The cyclic oligofuran according to claim 97, having the structure 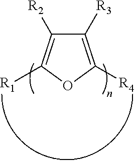
wherein
each of R₁ and R₄ is a point of connectivity to another furan of the cyclic oligofuran ring system,
R₂ and R₃ is selected from hydrogen; C₁-C₁₀alkyl; C₂-C₁₀alkenyl; C₂-C₁₀alkynyl; C₆-C₁₀aryl; C₅-C₁₀ heteroaryl comprising between 1 and 5 heteroatoms selected from N, O, S; —CN; —CO₂H; —OH; —SH; —NR′R″R′″; —NO₂; (C₁-C₁₀alkyl)₃Si—; (C₁-C₁₀alkyl)₃Sn—; trifluoromethanesulfonate (triflate) and halogen, wherein each of R′, R″ and R′″ may be the same or different and may be selected from hydrogen, C₁-C₁₀alkyl, C₂-C₁₀alkenyl, C₂-C₁₀alkynyl and C₆-C₁₀aryl; and
wherein n is an integer between 4 and 20.

99. The cyclic oligofuran according to claim 97, comprising 6 furan ring moieties, wherein each of the furan ring moieties is optionally substituted.
